(19) United States
(12) Patent Application Publication
(10) Pub. No.: US 2026/0364042 A1
(43) Pub. Date: Nov. 19, 2026

(54) ORAL COMPOSITIONS OF OMEGA-3 ETHYL ESTERS

(71) Applicant: Lazirex Corporation, Flowery Branch, GA (US)

(72) Inventors: Eric D. Rustin, Johns Creek, GA (US); Roy W. Bryant, Dahlonega, GA (US); H. Greg Thomas, Carrollton, GA (US); Courtney Rouse Smith, Gainesville, GA (US); Mary Thi Schuster, Auburn, GA (US)

(73) Assignee: Lazirex Corporation, Flowery Branch, GA (US)

(21) Appl. No.: 19/722,716

(22) Filed: Jun. 26, 2026

Related U.S. Application Data
(63) Continuation of application No. 19/542,524, filed on Feb. 17, 2026.
(60) Provisional application No. 63/759,934, filed on Feb. 18, 2025.

(51) Int. Cl. A61K 31/232; A61K 9/00; A61K 9/08; A61K 47/44

(52) U.S. Cl. CPC A61K 31/232; A61K 9/0053; A61K 9/08; A61K 47/44

(57) ABSTRACT

Oral compositions, including pharmaceutical compositions and dietary supplements, of a mixture of fatty acid esters comprising omega-3 fatty acid ethyl esters and a liquid carrier are provided herein. Methods of using the oral compositions to treat conditions and improve health are also provided.

### CROSS-REFERENCE TO RELATED APPLICATIONS

This application claims priority to U.S. Provisional Patent Application No. 63/759,934, filed Feb. 18, 2025, the entire contents of which are incorporated by referenced herein.

### BACKGROUND

Omega-3 fatty acid products are approved for use as an adjunct to diet to reduce triglyceride (TG) levels in adults with severe hypertriglyceridemia (e.g., TG levels of 500 mg/dL or more). Exemplary products include Lovaza® (omega-3-acid ethyl esters, GlaxoSmithKline), and generic versions of Lovaza® from various manufacturers. These products contain ethyl esters of the long-chain omega-3 fatty acids docosahexaenoic acid (DHA) and eicosapentaenoic acid (EPA) in liquid-filled gel capsules. Lovaza® capsules contain 0.465 g EPA and 0.375 g DHA in each 1 g capsule. Lovaza® capsules also contain 4 mg α-tocopherol (in a carrier of vegetable oils including soybean oil) and gelatin, glycerol, and purified water (components of the capsule shell) as inactive ingredients. The daily dose of Lovaza® comprises 4 grams per day taken as a single 4-gram dose (4 capsules) or as two 2-gram doses (2 capsules).

Patients with prescriptions for omega-3 ethyl ester products like Lovaza® may be redirected to over-the-counter (OTC) formulations that differ in dosage, purity, and fatty acid composition. One such OTC product is Nature Made® Fish Oil, with one dose defined as a single capsule per day that contains 720 mg omega-3 fatty acid ethyl esters. This dose is less than the therapeutically effective amount (3,360 mg per day) of the Lovaza® capsules indicated to reduce triglyceride levels. Additionally, the OTC products tend to be more impure and contain oils other than omega-3 fatty acid esters that are responsible for putrefaction (fishy smell) and eructation (burping).

Both the Lovaza® capsules and Nature Made® OTC capsules can be problematic or impossible to administer in patients with difficulty swallowing. Some patients report that the Nature Made® OTC capsules have a fishy taste and experience burping after consumption of the capsules.

Additionally, the capsule formulations contain non-trivial amounts of omega-6 fatty acid ethyl esters, which are pro-inflammatory, contrary to the anti-inflammatory properties of omega-3 fatty acid ethyl esters.

There remains a need for improved formulations of omega-3 fatty acid ethyl esters that are readily administered (e.g., easy to swallow), and/or contain a therapeutic dose of omega-3 fatty acid ethyl esters, and/or do not elicit a fishy smell or cause burping in patients.

### SUMMARY

In one aspect, the present disclosure provides oral compositions (e.g., solutions) comprising a mixture of fatty acid esters comprising omega-3 fatty acid ethyl esters and a liquid carrier comprising olive oil. In some embodiments, the oral composition is an oral pharmaceutical composition. In some embodiments, the oral composition is a dietary supplement.

In a second aspect, the present disclosure provides methods of administering omega-3 fatty acid ethyl esters, comprising orally administering to a subject an oral composition as described herein.

In a third aspect, the present disclosure provides methods of reducing triglyceride (TG) levels in a subject in need thereof, comprising orally administering to the subject a therapeutically effective dose of the oral composition described herein.

In a fourth aspect, the present disclosure provides methods of providing cognitive support in a subject in need thereof, comprising orally administering to the subject a therapeutically effective dose of the oral composition described herein.

In a fifth aspect, the present disclosure provides methods of providing mood support in a subject in need thereof, comprising orally administering to the subject a therapeutically effective dose of the oral composition described herein.

In a sixth aspect, the present disclosure provides methods of improving heart health in a subject in need thereof, comprising orally administering to the subject a therapeutically effective dose of the oral composition described herein.

In a seventh aspect, the present disclosure provides methods of improving joint health in a subject in need thereof, comprising orally administering to the subject a therapeutically effective dose of the oral composition described herein.

In an eighth aspect, the present disclosure provides methods of improving eye health in a subject in need thereof, comprising orally administering to the subject a therapeutically effective dose of the oral composition described herein.

In a ninth aspect, the present disclosure provides methods of providing thermoregulation support in a subject in need thereof, comprising orally administering to the subject a therapeutically effective dose of the oral composition described herein.

In a tenth aspect, the present disclosure provides methods of providing hormonal balance support in a subject in need thereof, comprising orally administering to the subject a therapeutically effective dose of the oral composition described herein.

### BRIEF DESCRIPTION OF THE DRAWINGS

These and other features, aspects, and advantages of the present disclosure will become better understood with regard to the following description, and accompanying drawings, where:

FIG. 1 illustrates the results of the color analysis for samples of an oral solution of the present disclosure housed at room temperature (25 °C) and within containers in vertical/upright (“upr”) and horizontal (“horiz”) static positions. The color analysis was carried out over 24 months. Color was measured by UV-VIS spectrophotometry at 400 nm. All results were within the specification of NMT 0.75.

FIG. 2 illustrates the Acid Value for samples of an oral solution of the present disclosure housed at room temperature (25 °C) and within containers in upr and horiz static positions. The Acid Value test was carried out over 24 months. The Acid Value results ranged from 0.5-1.5 for samples at all conditions and conform to the specification of NMT 2.0.

FIG. 3 illustrates the Peroxide Value (PV) for samples of an oral solution of the present disclosure housed at room temperature (25 °C) and within containers in upr and horiz static positions. The PV test was carried out over 24 months. The PV results ranged from 2-4 for the samples at all conditions and conform to the specification of NMT 10 meq/kg.

FIG. 4 illustrates the Anisidine Value (AV) for samples of an oral solution of the present disclosure housed at room temperature (25 °C) and within containers in upr and horiz static positions. The AV test was carried out over 24 months. The AV results ranged from 38.6-52.0 for the 25 °C samples and conform to the specification of NMT 80.

FIG. 5 illustrates the methylparaben content for samples of an oral solution of the present disclosure housed at room temperature (25 °C) and within containers in upr and horiz static positions. The methylparaben content was measured over 24 months. The methylparaben content ranged from 97.5%-99.2% for the 25 °C samples and conform to the specification of a methylparaben content from 90.0%-110.0%.

FIG. 6 illustrates the propylparaben content for samples of an oral solution of the present disclosure housed at room temperature (25 °C) and within containers in upr and horiz static positions. The propylparaben content was measured over 24 months. The propylparaben content ranged from 95.4%-99.3% for the 25 °C samples and conform to the specification of a propylparaben content from 90.0%-110.0%.

FIG. 7 illustrates EPA n-3 EE and DHA n-3 EE accelerated aging of an oral solution of the present disclosure housed within containers in vertical/upright (“upr”) and horizontal (“horiz”) static positions. The trend line demonstrates omega-3 EE composition stability beyond the 24-month shelf-life projection.

FIG. 8 illustrates omega-3 EE composition accelerated aging of an oral solution of the present disclosure housed within containers in vertical/upright (“upr”) and horizontal (“horiz”) static positions. The trend line demonstrates omega-3 EE composition stability beyond the 24-month shelf-life projection.

### DETAILED DESCRIPTION

Described herein are oral liquid compositions of omega-3 fatty acid ethyl esters, therapeutic methods of using them (e.g., for reducing triglyceride levels), and methods of using them for health benefits (e.g., cognitive support, mood support, heart health, joint health, eye health, bone health, thermoregulation support, and hormonal balance support). As described in more detail below, the present inventors determined that high dosages of omega-3 fatty acid ethyl esters can be formulated as an oral liquid composition with appropriate selection of excipients, e.g., liquid carrier (refined olive oil) and antioxidants. The oral compositions exhibit advantageous physicochemical properties, including good physical stability (e.g., stability against color change and weight loss). The oral compositions can be administered as a single dose and provide a higher potency of the omega-3 fatty acid ethyl esters compared to known supplements that are in the form of a capsule. Due to the high potency of the oral composition of the present disclosure, one dose may provide the equivalent amount of omega-3 fatty acid ethyl esters as 4 large, difficult to swallow soft-gel capsules currently on the market. Additionally, when the oral composition is in the form of a solution, absorption of the omega-3 fatty acid ethyl esters is superior to when omega-3 fatty acid ethyl esters are administered in soft-gel capsules. Patients administered the oral composition of the present disclosure may experience diminished “pill fatigue”, where the patient experiences the mental and physical burden of being administered one or more large pills, resulting in reduced motivation and compliance. The oral composition of the present disclosure has a refreshing taste (e.g., lemon or citrus taste) and does not have a fishy taste or result in a fishy aftertaste or burping. Administration of the oral composition of the present disclosure provides several health benefits including one or more of: reduction in triglyceride levels, cognitive support (e.g., mental clarity, focus, memory support), mood support (e.g., helps maintain a balanced mood and emotional well-being), heart health (e.g., supports cardiovascular function), joint health (e.g., helps maintain joint comfort through healthy inflammation balance), eye health (e.g., supports eye hydration), bone health (e.g., provides fatty acids that support overall bone health), thermoregulation support (e.g., supports healthy temperature regulation during mid-life transitions), and hormonal balance support (e.g., supports hormonal balance and healthy inflammatory responses).

### DEFINITIONS

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

“About,” as used herein, will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

“EPA n-3 EE” refers to “eicosapentaenoic acid ethyl ester”, the structure of which is shown below and is an omega-3 fatty acid ester:

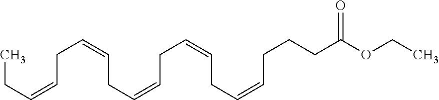 [SMILES: CC/C=C\C/C=C\C/C=C\C/C=C\C/C=C\CCCC(=O)OCC]

“DHA n-3 EE” refers to “docosahexaenoic acid ethyl ester”, the structure of which is shown below and is an omega-3 fatty acid ester:

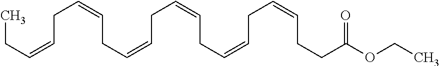 [SMILES: CC/C=C\C/C=C\C/C=C\C/C=C\C/C=C\C/C=C\CCC(=O)OCC]

“ALA n-3 EE” refers to “alpha-linolenic acid ethyl ester”, the structure of which is shown below and is an omega-3 fatty acid ester:

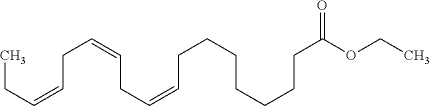 [SMILES: CC/C=C\C/C=C\C/C=C\CCCCCCCC(=O)OCC]

“SDA n-3 EE” refers to “stearidonic acid ethyl ester”, also known as “moroctic acid”, the structure of which is shown below and is an omega-3 fatty acid ester:

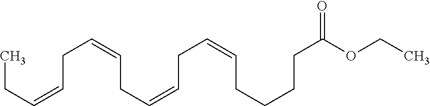 [SMILES: CC/C=C\C/C=C\C/C=C\C/C=C\CCCCC(=O)OCC]

“ETA n-3 EE” refers to “eicosatetraenoic acid ethyl ester”, the structure of which is shown below and is an omega-3 fatty acid ester:

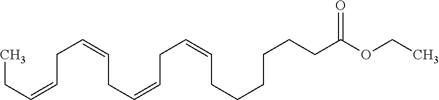 [SMILES: CC/C=C\C/C=C\C/C=C\C/C=C\CCCCCCC(=O)OCC]

“HPA n-3 EE” refers to “heneicosapentaenoic acid ethyl ester”, the structure of which is shown below and is an omega-3 fatty acid ester:

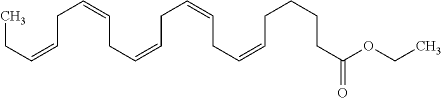 [SMILES: CC/C=C\C/C=C\C/C=C\C/C=C\C/C=C\CCCCC(=O)OCC]

“DPA n-3 EE” refers to “docosapentaenoic acid ethyl ester”, the structure of which is shown below and is an omega-3 fatty acid ester:

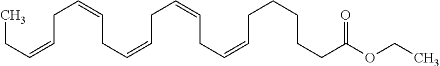 [SMILES: CC/C=C\C/C=C\C/C=C\C/C=C\C/C=C\CCCCCC(=O)OCC]

“ETA n-6 EE” refers to “eicosatetraenoic acid ethyl ester”, known equivalently as “arachidonic acid ethyl ester” and designated as “ARA n-6 EE”, the structure of which is shown below and is an omega-6 fatty acid ester:

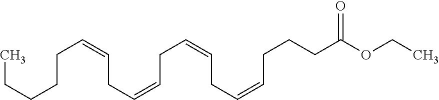 [SMILES: CCCCC/C=C\C/C=C\C/C=C\C/C=C\CCCC(=O)OCC]

“DPA n-6 EE” refers to “docosapentaenoic acid ethyl ester”, the structure of which is shown below and is an omega-6 fatty acid ester:

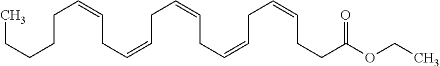 [SMILES: CCCCC/C=C\C/C=C\C/C=C\C/C=C\C/C=C\CCC(=O)OCC]

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds, and the like. In certain embodiments, the subject is a primate. In some embodiments, the subject is a human.

A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses.

As used herein, “omega-3 fatty acid ethyl ester” refers to the conventional nomenclature for such compounds, i.e., an ethyl ester of a polyunsaturated fatty acid, wherein the first double bond occurs between the third and fourth carbons (n-3) from the methyl end of the molecule (omega position). The two primary omega-3 fatty acid acids are eicosapentaenoic acid (EPA, 20: 5n-3) and docosahexaenoic acid (DHA, 22: 6n-3), both of which are derived from fish. These chemical compounds are converted during processing and purification steps to their corresponding ethyl esters (EPA n-3 EE and DPA n-3 EE, respectively). These compounds are equivalent to their alternative designation as (EPA EE and DPA EE). The five minor omega-3 fatty acid ethyl esters include stearidonic (moroctic) acid ethyl ester (SDA n-3 EE), heneicosapentaenoic acid ethyl ester (HPA n-3 EE), docosapentanenoic acid ethyl ester (DPA n-3 EE), eicosatetraenoic acid ethyl ester (ETA n-3 EE), and alpha-linolenic acid (ALA n-3 EE). As used herein, “total omega-3 fatty acid ethyl esters” refers to the sum of EPA n-3 EE, DHA n-3 EE, SDA n-3 EE, HPA n-3 EE, DPA n-3 EE, ETA n-3 EE, and ALA n-3 EE.

Similarly, as used herein, “omega-6 fatty acid ethyl ester” refers to an ethyl ester of a polyunsaturated fatty acid, wherein the first double bond occurs between the sixth and seventh carbons (n-6) from the methyl end of the molecule (omega position).

As used herein, “other ethyl esters” refers to ethyl esters of compounds that are not one of the seven primary omega-3-fatty acid ethyl esters identified above (i.e., EPA n-3 EE, DHA n-3 EE, SDA n-3 EE, HPA n-3 EE, DPA n-3 EE, ETA n-3 EE, and ALA n-3 EE).

As used herein, the phrase “per 10 mL” or “/10 mL” with respect to a particular substance (e.g., “total omega 3-fatty acid ethyl esters per 10 mL” or “total omega-3 fatty acid ethyl esters per 10 mL”) refers to the concentration of the substance in the oral composition, not that the particular composition has a volume of 10 mL. A person of ordinary skill in the art can calculate the amount of a substance in any volume of oral composition from the given concentration of a substance per 10 mL. For example, the concentration of substance Y is the same in (1) oral composition A containing X amount of substance Y per 10 mL and (2) oral composition B containing 0.5X amount of substance Y per 5 mL.

As used herein, the phrase “therapeutic agent” refers to any agent that has a therapeutic effect and/or elicits a desired biological and/or pharmacological effect, when administered to a subject.

As used herein, the term “therapeutically effective,” with respect to an amount (e.g., “therapeutically effective amount”) or dose (e.g., “a therapeutically effective dose”) refers to an amount or dose of a therapeutic agent (e.g., omega-3 fatty acid ethyl esters) that confers a therapeutic effect in a target patient population. The therapeutic effect may be objective (i.e., measurable by some test or marker) or subjective (i.e., subject gives an indication of or feels an effect). A therapeutically effective amount or therapeutically effective dose may be an amount effective to treat, ameliorate, or prevent a disease or condition, or to exhibit a detectable therapeutic or preventive effect, such as by ameliorating symptoms associated with the disease, preventing or delaying the onset of the disease or condition, and/or also lessening the severity or frequency of symptoms of the disease or condition. It should be understood that a therapeutically effective amount or therapeutically effective dose may not necessarily be effective in every subject treated with that amount. A therapeutically effective amount may be administered in a dosing regimen that comprises multiple doses. For any particular therapeutic agent, a therapeutically effective amount (and/or an appropriate unit dose within an effective dosing regimen) may vary, for example, depending on route of administration, or use in combination with other pharmaceutical agents. Also, the specific therapeutically effective amount or therapeutically effective dose for any particular subject or patient population may depend upon a variety of factors including the disorder being treated and the severity of the disorder; the activity of the specific therapeutic agent employed; the specific composition employed; the age, body weight, general health, sex and diet of the subject; the time of administration, route of administration, and/or rate of excretion or metabolism of the specific therapeutic agent employed; the duration of the treatment; and like factors as is well known in the medical arts.

As used herein, the term “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically (e.g., through stabilization of a discernible symptom), physiologically, (e.g., through stabilization of a physical parameter), or both. In yet another embodiment, “treat,” “treating,” or “treatment” refers to preventing or delaying the onset or development or progression of the disease or disorder.

As used herein, a subject is “in need of” a treatment if such subject would benefit biologically, medically or in quality of life from such treatment.

### Compositions

In one aspect of the present disclosure, an oral composition comprising omega-3 fatty acid ethyl esters is provided. In some embodiments, the oral composition is in the form of a solution. In other embodiments, the oral composition is in the form of a suspension. In some embodiments, the oral composition comprises omega-3 fatty acid ethyl esters and omega-6 fatty acid ethyl esters. In some embodiments, the oral composition comprises a liquid carrier. In some embodiments, provided herein is an oral composition comprising a mixture of fatty acid esters comprising omega-3 fatty acid ethyl esters and omega-6 fatty acid ethyl esters, and a liquid carrier comprising olive oil.

In some embodiments, the oral composition is an oral pharmaceutical composition that treats certain disorders for which omega-3 fatty acids are indicated, e.g., reducing triglycerides in a subject.

In some embodiments, the oral composition is in the form of a dietary supplement that provides real or perceived health benefits in a subject, e.g., cognitive support, mood support, heart health, joint health, eye health, bone health, thermoregulation support, and hormonal balance support.

### Fatty Acid Esters

In some embodiments, omega-3 fatty acid ethyl esters of the present disclosure comprise one or more of eicosapentaenoic acid ethyl ester (EPA n-3 EE), docosahexaenoic acid ethyl ester (DHA n-3 EE), stearidonic (moroctic) acid ethyl ester (SDA n-3 EE), heneicosapentaenoic acid ethyl ester (HPA n-3 EE), docosapentaenoic acid ethyl ester (DPA n-3 EE), eicosatetraenoic acid ethyl ester (ETA n-3 EE), and alpha-linolenic acid (ALA n-3 EE).

In some embodiments, omega-3 fatty acid ethyl esters of the present disclosure comprise EPA n-3 EE and DHA n-3 EE as major components and one or more of SDA n-3 EE, HPA n-3 EE, DPA n-3 EE, ETA n-3 EE, and ALA n-3 EE as minor components.

In some embodiments, the mixture of fatty acid esters comprises at least 90% by weight of EPA n-3 EE, DHA n-3 EE, SDA n-3 EE, HPA n-3 EE, DPA n-3 EE, ETA n-3 EE, and ALA n-3 EE; such as, for example, at least 91%, at least 92%, at least 93%, at least 94%, at least 95%, at least 96%, at least 97%, at least 98%, or at least 99%.

In some embodiments, the mixture of fatty acid esters comprises at least 90% by weight of EPA n-3 EE, DHA n-3 EE, SDA n-3 EE, HPA n-3 EE, DPA n-3 EE, ETA n-3 EE, and ALA n-3 EE; wherein EPA n-3 EE and DHA n-3 EE are present as major components and one or more of SDA n-3 EE, HPA n-3 EE, DPA n-3 EE, ETA n-3 EE, and ALA n-3 EE are present as minor components.

In some embodiments, the oral composition of the present disclosure (e.g., an oral pharmaceutical composition or dietary supplement) comprises at least 1,500 mg total omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE) per 10 mL, such as at least 1,750 mg omega-3 fatty acid ethyl esters per 10 mL, at least 2,000 mg omega-3 fatty acid ethyl esters, or at least 2,500 mg omega-3 fatty acid ethyl esters per 10 mL.

In some embodiments, the oral composition comprises at least 3,000 mg total omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE) per 10 mL, such as at least 3,500 mg omega-3 fatty acid ethyl esters per 10 mL, at least 3,600 mg omega-3 fatty acid ethyl esters, or at least 3,750 mg omega-3 fatty acid ethyl esters per 10 mL.

In some embodiments, the oral composition comprises at least 3,000 mg total omega-3 fatty acid ethyl esters per 10 mL. In some embodiments, the oral composition comprises at least 3,100 mg total omega-3 fatty acid ethyl esters per 10 mL. In some embodiments, the oral composition comprises at least 3,200 mg total omega-3 fatty acid ethyl esters per 10 mL. In some embodiments, the oral composition comprises at least 3,300 mg total omega-3 fatty acid ethyl esters per 10 mL. In some embodiments, the oral composition comprises at least 3,400 mg total omega-3 fatty acid ethyl esters per 10 mL. In some embodiments, the oral composition comprises at least 3,500 mg total omega-3 fatty acid ethyl esters per 10 mL. In some embodiments, the oral composition comprises at least 3,600 mg total omega-3 fatty acid ethyl esters per 10 mL. In some embodiments, the oral composition comprises at least 3,700 mg total omega-3 fatty acid ethyl esters per 10 mL. In some embodiments, the oral composition comprises at least 3,750 mg total omega-3 fatty acid ethyl esters per 10 mL. In some embodiments, the oral composition comprises at least 3,800 mg total omega-3 fatty acid ethyl esters per 10 mL. In some embodiments, the oral composition comprises at least 3,900 mg total omega-3 fatty acid ethyl esters per 10 mL. In some embodiments, the oral composition comprises at least 4,000 mg total omega-3 fatty acid ethyl esters per 10 mL. In some embodiments, the oral composition comprises at least 4,100 mg total omega-3 fatty acid ethyl esters per 10 mL. In some embodiments, the oral composition comprises at least 4200 mg total omega-3 fatty acid ethyl esters per 10 mL.

In some embodiments, the oral composition comprises from 3,000 mg to 5,000 mg total omega-3 fatty acid ethyl esters per 10 mL, such as from 3,200 to 4,800 mg total omega-3 fatty acid ethyl esters per 10 mL, from 3,500 to 4,000 mg total omega-3 fatty acid ethyl esters per 10 mL, or from 3,750 to 5,000 mg total omega-3 fatty acid ethyl esters per 10 mL.

In some embodiments, the oral composition comprises from 3,000 mg to 5,000 mg total omega-3 fatty acid ethyl esters per 10 mL. In some embodiments, the oral composition comprises from 3,000 mg to 4,800 mg total omega-3 fatty acid ethyl esters per 10 mL. In some embodiments, the oral composition comprises from 3,000 mg to 4,600 mg total omega-3 fatty acid ethyl esters per 10 mL. In some embodiments, the oral composition comprises from 3,000 mg to 4,500 mg total omega-3 fatty acid ethyl esters per 10 mL. In some embodiments, the oral composition comprises from 3,000 mg to 4,400 mg total omega-3 fatty acid ethyl esters per 10 mL. In some embodiments, the oral composition comprises from 3,000 mg to 4,200 mg total omega-3 fatty acid ethyl esters per 10 mL. In some embodiments, the oral composition comprises from 3,000 mg to 4,000 mg total omega-3 fatty acid ethyl esters per 10 mL. In some embodiments, the oral composition comprises from 3,000 mg to 3,800 mg total omega-3 fatty acid ethyl esters per 10 mL. In some embodiments, the oral composition comprises from 3,000 mg to 3,600 mg total omega-3 fatty acid ethyl esters per 10 mL. In some embodiments, the oral composition comprises from 3,000 mg to 3,400 mg total omega-3 fatty acid ethyl esters per 10 mL. In some embodiments, the oral composition comprises from 3,000 mg to 3,200 mg total omega-3 fatty acid ethyl esters per 10 mL.

In some embodiments, the oral composition comprises from 3,200 mg to 5,000 mg total omega-3 fatty acid ethyl esters per 10 mL. In some embodiments, the oral composition comprises from 3,400 mg to 5,000 mg total omega-3 fatty acid ethyl esters per 10 mL. In some embodiments, the oral composition comprises from 3,600 mg to 5,000 mg total omega-3 fatty acid ethyl esters per 10 mL. In some embodiments, the oral composition comprises from 3,800 mg to 5,000 mg total omega-3 fatty acid ethyl esters per 10 mL. In some embodiments, the oral composition comprises from 3,900 mg to 5,000 mg total omega-3 fatty acid ethyl esters per 10 mL. In some embodiments, the oral composition comprises from 4,000 mg to 5,000 mg total omega-3 fatty acid ethyl esters per 10 mL. In some embodiments, the oral composition comprises from 4,200 mg to 5,000 mg total omega-3 fatty acid ethyl esters per 10 mL. In some embodiments, the oral composition comprises from 4,400 mg to 5,000 mg total omega-3 fatty acid ethyl esters per 10 mL. In some embodiments, the oral composition comprises from 4,600 mg to 5,000 mg total omega-3 fatty acid ethyl esters per 10 mL.

In some embodiments, the oral composition comprises at least 3,600 mg/10 mL of the sum of EPA n-3 EE, DHA n-3 EE, SDA n-3 EE, HPA n-3 EE, DPA n-3 EE, ETA n-3 EE, and ALA n-3 EE, such as, for example, from 3,600 mg/10 mL to 5,000 mg/10 mL, from 3,600 mg/10 mL to 4,500 mg/10 mL, from 3,600 mg/10 mL to 4,250 mg/10 mL, or from 3,600 mg/10 mL to 4,000 mg/10 mL.

In some embodiments, the oral composition comprises at least 1,500 mg of the sum of EPA n-3 EE and DHA n-3 EE per 10 mL, such as, for example, from 1,750 mg to 5,000 mg per 10 mL; 2,000 mg to 5,000 mg per 10 mL; 2,500 mg to 5,000 mg per 10 mL; 3,000 mg to 5,000 mg per 10 mL; from 3,000 mg to 4,750 mg per 10 mL; from 3,000 mg to 4,500 mg per 10 mL; from 3,000 mg to 4,250 mg per 10 mL; from 3,100 mg to 5,000 mg per 10 mL; from 3,100 mg to 4,750 mg per 10 mL; from 3,100 mg to 4,500 mg per 10 mL; from 3,100 mg to 4,250 mg per 10 mL; from 3,200 mg to 5,000 mg per 10 mL; from 3,200 mg to 4,750 mg per 10 mL; from 3,200 mg to 4,500 mg per 10 mL; from 3,200 mg to 4,250 mg per 10 mL; from 3,300 mg to 5,000 mg per 10 mL; from 3,300 mg to 4,750 mg per 10 mL; from 3,300 mg to 4,500 mg per 10 mL; from 3,300 mg to 4,250 mg per 10 mL; from 3,400 mg to 5,000 mg per 10 mL; from 3,400 mg to 4,750 mg per 10 mL; from 3,400 mg to 4,500 mg per 10 mL; from 3,400 mg to 4,250 mg per 10 mL; from 3,500 mg to 5,000 mg per 10 mL; from 3,500 mg to 4,750 mg per 10 mL; from 3,500 mg to 4,500 mg per 10 mL; from 3,500 mg to 4,250 mg per 10 mL; from 3,600 mg to 5,000 mg per 10 mL; from 3,600 mg to 4,750 mg per 10 mL; from 3,600 mg to 4,500 mg per 10 mL; from 3,600 mg to 4,250 mg per 10 mL; from 3,700 mg to 5,000 mg per 10 mL; from 3,700 mg to 4,750 mg per 10 mL; from 3,700 mg to 4,500 mg per 10 mL; from 3,700 mg to 4,250 mg per 10 mL; from 3,800 mg to 5,000 mg per 10 mL; from 3,800 mg to 4,750 mg per 10 mL; from 3,800 mg to 4,500 mg per 10 mL; from 3,800 mg to 4,250 mg per 10 mL; from 3,900 mg to 5,000 mg per 10 mL; from 3,900 mg to 4,750 mg per 10 mL; from 3,900 mg to 4,500 mg per 10 mL; from 3,900 mg to 4,250 mg per 10 mL; from 4,000 mg to 5,000 mg per 10 mL; from 4,000 mg to 4,750 mg per 10 mL; from 4,000 mg to 4,500 mg per 10 mL; or from 4,000 mg to 4,250 mg per 10 mL. In some embodiments, the oral composition comprises a sum of EPA n-3 EE and DHA n-3 EE per 10 mL from 1,500 mg to 5,000 mg per 10 mL, e.g., from 1,500 mg to 4,750 mg per 10 mL; from 1,500 mg to 4,500 mg per 10 mL; from 1,500 mg to 4,250 mg per 10 mL; 1,500 mg to 4,000 mg per 10 mL; from 1,500 mg to 3,750 mg per 10 mL; from 1,500 mg to 3,500 mg per 10 mL; from 1,500 mg to 3,250 mg per 10 mL; from 1,500 mg to 3,000 mg per 10 mL; from 2,000 mg to 5,000 mg per 10 mL; or from 2,000 mg to 4,500 mg per 10 mL.

In some embodiments, the oral composition comprises at least 3,000 mg of the sum of EPA n-3 EE and DHA n-3 EE per 10 mL, such as from 3,000 mg to 5,000 mg per 10 mL. In some embodiments, the oral composition comprises at least 3,000 mg of the sum of EPA n-3 EE and DHA n-3 EE per 10 mL, such as from 3,000 mg to 4,000 mg per 10 mL.

In some embodiments, the oral composition comprises at least 3,100 mg of the sum of EPA n-3 EE and DHA n-3 EE per 10 mL, such as from 3,100 mg to 5,000 mg per 10 mL. In some embodiments, the oral composition comprises at least 3,100 mg of the sum of EPA n-3 EE and DHA n-3 EE per 10 mL, such as from 3,100 mg to 4,000 mg per 10 mL.

In some embodiments, the oral composition comprises at least 3,200 mg of the sum of EPA n-3 EE and DHA n-3 EE per 10 mL, such as from 3,200 mg to 5,000 mg per 10 mL. In some embodiments, the oral composition comprises at least 3,200 mg of the sum of EPA n-3 EE and DHA n-3 EE per 10 mL, such as from 3,200 mg to 4,000 mg per 10 mL.

In some embodiments, the oral composition comprises at least 3,300 mg of the sum of EPA n-3 EE and DHA n-3 EE per 10 mL, such as from 3,300 mg to 5,000 mg per 10 mL. In some embodiments, the oral composition comprises at least 3,300 mg of the sum of EPA n-3 EE and DHA n-3 EE per 10 mL, such as from 3,300 mg to 4,000 mg per 10 mL.

In some embodiments, the oral composition comprises at least 3,400 mg of the sum of EPA n-3 EE and DHA n-3 EE per 10 mL, such as from 3,400 mg to 5,000 mg per 10 mL. In some embodiments, the oral composition comprises at least 3,400 mg of the sum of EPA n-3 EE and DHA n-3 EE per 10 mL, such as from 3,400 mg to 4,000 mg per 10 mL.

In some embodiments, the oral composition comprises at least 3,500 mg of the sum of EPA n-3 EE and DHA n-3 EE per 10 mL, such as from 3,500 mg to 5,000 mg per 10 mL. In some embodiments, the oral composition comprises at least 3,500 mg of the sum of EPA n-3 EE and DHA n-3 EE per 10 mL, such as from 3,500 mg to 4,000 mg per 10 mL.

In some embodiments, the oral composition comprises at least 3,600 mg of the sum of EPA n-3 EE and DHA n-3 EE per 10 mL, such as from 3,600 mg to 5,000 mg per 10 mL. In some embodiments, the oral composition comprises at least 3,600 mg of the sum of EPA n-3 EE and DHA n-3 EE per 10 mL, such as from 3,600 mg to 4,000 mg per 10 mL.

In some embodiments, the oral composition comprises at least 3,700 mg of the sum of EPA n-3 EE and DHA n-3 EE per 10 mL, such as from 3,700 mg to 5,000 mg per 10 mL. In some embodiments, the oral composition comprises at least 3,700 mg of the sum of EPA n-3 EE and DHA n-3 EE per 10 mL, such as from 3,700 mg to 4,000 mg per 10 mL.

In some embodiments, the oral composition comprises at least 3,800 mg of the sum of EPA n-3 EE and DHA n-3 EE per 10 mL such as from 3,800 mg to 5,000 mg per 10 mL. In some embodiments, the oral composition comprises at least 3,800 mg of the sum of EPA n-3 EE and DHA n-3 EE per 10 mL, such as from 3,800 mg to 4,000 mg per 10 mL.

In some embodiments, the oral composition comprises at least 3,900 mg of the sum of EPA n-3 EE and DHA n-3 EE per 10 mL, such as from 3,900 mg to 5,000 mg per 10 mL. In some embodiments, the oral composition comprises at least 3,900 mg of the sum of EPA n-3 EE and DHA n-3 EE per 10 mL, such as from 3,900 mg to 4,000 mg per 10 mL.

In some embodiments, the oral composition comprises at least 4,000 mg of the sum of EPA n-3 EE and DHA n-3 EE per 10 mL, such as from 4,000 mg to 5,000 mg per 10 mL.

In any of the embodiments described herein, the concentration of the total omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE) remains the same for different dosing volumes. For example, a person of ordinary skill in the art would understand that an oral composition comprising 5,000 mg of the sum of EPA n-3 EE and DHA n-3 EE per 10 mL is equal in concentration to an oral composition comprising 2,500 mg of the sum of EPA n-3 EE and DHA n-3 EE per 5 mL and an oral composition comprising 500 mg of the sum of EPA n-3 EE and DHA n-3 EE per 1 mL. The concentration of the total omega-3 fatty acid ethyl esters in the oral composition can be determined accordingly.

In some embodiments, the sum of EPA n-3 EE and DHA n-3 EE comprises at least 60% by weight of the total omega-3 fatty acid ethyl esters, such as at least 65% by weight of the total omega-3 fatty acid ethyl esters, or at least 70% by weight of the total omega-3 fatty acid ethyl esters. In some embodiments, the sum of EPA n-3 EE and DHA n-3 EE comprises at least 70% by weight of the total omega-3 fatty acid ethyl esters, such as at least 75% by weight of the total omega-3 fatty acid ethyl esters, or at least 80% by weight of the total omega-3 fatty acid ethyl esters. In some embodiments, the sum of EPA n-3 EE and DHA n-3 EE comprises at least 80% by weight of the total omega-3 fatty acid ethyl esters, such as at least 85% by weight of the total omega-3 fatty acid ethyl esters, or at least 90% by weight of the total omega-3 fatty acid ethyl esters. In some embodiments, the sum of EPA n-3 EE and DHA n-3 EE comprises at least 85% by weight of the total omega-3 fatty acid ethyl esters. In some embodiments, the sum of EPA n-3 EE and DHA n-3 EE comprises at least 90% by weight of the total omega-3 fatty acid ethyl esters. In some embodiments, the sum of EPA n-3 EE and DHA n-3 EE comprises at least 95% by weight of the total omega-3 fatty acid ethyl esters.

In some embodiments, the ratio of EPA n-3 EE to DHA n-3 EE is from 5:1 to 1:5, such as from 4:1 to 1:4, from 3:1 to 1:3, from 2:1 to 1:2, or from 2:1 to 1:1. In some embodiments, the ratio of EPA n-3 EE to DHA n-3 EE is from 5:1 to 1:5. In some embodiments, the ratio of EPA n-3 EE to DHA n-3 EE is from 4:1 to 1:4. In some embodiments, the ratio of EPA n-3 EE to DHA n-3 EE is from 3:1 to 1:3. In some embodiments, the ratio of EPA n-3 EE to DHA n-3 EE is from 2:1 to 1:2. In some embodiments, the ratio of EPA n-3 EE to DHA n-3 EE is from 1.5:1 to 1:1. In some embodiments, the ratio of EPA n-3 EE to DHA n-3 EE is from 1.3:1 to 1:1. In some embodiments, the ratio of EPA n-3 EE to DHA n-3 EE is from 1.2:1 to 1:1. In some embodiments, the ratio of EPA n-3 EE to DHA n-3 EE is 1.3:1. In some embodiments, the ratio of EPA n-3 EE to DHA n-3 EE is 5:1. In some embodiments, the ratio of EPA n-3 EE to DHA n-3 EE is 4:1. In some embodiments, the ratio of EPA n-3 EE to DHA n-3 EE is 3:1. In some embodiments, the ratio of EPA n-3 EE to DHA n-3 EE is 2:1. In some embodiments, the ratio of EPA n-3 EE to DHA n-3 EE is 1:1. In some embodiments, the ratio of EPA n-3 EE to DHA n-3 EE is 1:2. In some embodiments, the ratio of EPA n-3 EE to DHA n-3 EE is 1:3. In some embodiments, the ratio of EPA n-3 EE to DHA n-3 EE is 1:4. In some embodiments, the ratio of EPA n-3 EE to DHA n-3 EE is 1:5.

In some embodiments, the oral composition of the present disclosure (e.g., oral pharmaceutical composition or dietary supplement) comprises a sum of the five minor omega-3 fatty acid ethyl esters (i.e., SDA n-3 EE, HPA n-3 EE, DPA n-3 EE, ETA n-3 EE, and ALA n-3 EE) in an amount less than 650 mg/10 mL, 600 mg/10 mL, 550 mg/10 mL, 500 mg/10 mL, 450 mg/10 mL, 400 mg/10 mL, such as, for example, less than 350 mg/10 mL, less than 300 mg/10 mL, less than 250 mg/10 mL, less than 200 mg/10 mL, less than 150 mg/10 mL, or less than 100 mg/10 mL. In some embodiments, the oral composition comprises a sum of minor omega-3 fatty acid ethyl esters (i.e., SDA n-3 EE, HPA n-3 EE, DPA n-3 EE, ETA n-3 EE, and ALA n-3 EE) in an amount from 100 to 400 mg/10 mL, such as, for example, from 150 to 400 mg/10 mL, from 175 to 400 mg/10 mL, from 200 to 400 mg/10 mL, from 225 to 400 mg/10 mL, from 250 to 400 mg/10 mL, from 275 to 400 mg/10 mL, from 300 to 400 mg/10 mL, from 325 to 400 mg/10 mL, from 350 to 400 mg/10 mL, or from 375 to 400 mg/10 mL.

In some embodiments, the oral composition of the present disclosure (e.g., oral pharmaceutical composition or dietary supplement) comprises less than 6% omega-6 fatty acid ethyl esters relative to total omega-3 fatty acid ethyl esters. In some embodiments, the oral composition comprises less than 5% omega-6 fatty acid ethyl esters relative to the total omega-3 fatty acid ethyl esters. In some embodiments, the oral composition comprises less than 4% omega-6 fatty acid ethyl esters relative to the total omega-3 fatty acid ethyl esters. In some embodiments, the oral composition comprises less than 3% omega-6 fatty acid ethyl esters relative to the total omega-3 fatty acid ethyl esters. In some embodiments, the oral composition comprises less than 2.5% omega-6 fatty acid ethyl esters relative to the total omega-3 fatty acid ethyl esters, such as less than 1.5%. In some embodiments, the oral composition comprises less than 2.25% omega-6 fatty acid ethyl esters relative to the total omega-3 fatty acid ethyl esters. In some embodiments, the oral composition comprises less than 2% omega-6 fatty acid ethyl esters relative to the total omega-3 fatty acid ethyl esters. In some embodiments, the oral composition comprises less than 1.75% omega-6 fatty acid ethyl esters relative to the total omega-3 fatty acid ethyl esters. In some embodiments, the oral composition comprises less than 1.5% omega-6 fatty acid ethyl esters relative to the total omega-3 fatty acid ethyl esters. In some embodiments, the oral composition comprises less than 1.25% omega-6 fatty acid ethyl esters relative to the total omega-3 fatty acid ethyl esters. In some embodiments, the oral composition comprises less than 1% omega-6 fatty acid ethyl esters relative to the total omega-3 fatty acid ethyl esters. In some embodiments, the oral composition comprises less than 0.75% omega-6 fatty acid ethyl esters relative to the total omega-3 fatty acid ethyl esters. In some embodiments, the oral composition comprises less than 0.5% omega-6 fatty acid ethyl esters relative to the total omega-3 fatty acid ethyl esters. In some embodiments, the oral composition comprises less than 0.25% omega-6 fatty acid ethyl esters relative to the total omega-3 fatty acid ethyl esters.

In some embodiments, the oral composition comprises less than 6% eicosatetraenoic acid ethyl ester (ETA n-6 EE) relative to the total omega-3 fatty acid ethyl esters. In some embodiments, the oral composition comprises less than 5% ETA n-6 EE relative to the total omega-3 fatty acid ethyl esters. In some embodiments, the oral composition comprises less than 4% ETA n-6 EE relative to the total omega-3 fatty acid ethyl esters. In some embodiments, the oral composition comprises less than 3% ETA n-6 EE relative to the total omega-3 fatty acid ethyl esters. In some embodiments, the oral composition comprises less than 2.5% eicosatetraenoic acid ethyl ester (ETA n-6 EE) relative to the total omega-3 fatty acid ethyl esters, such as less than 1%. In some embodiments, the oral composition comprises less than 2.25% ETA n-6 EE relative to the total omega-3 fatty acid ethyl esters. In some embodiments, the oral composition comprises less than 2% ETA n-6 EE relative to the total omega-3 fatty acid ethyl esters. In some embodiments, the oral composition comprises less than 1.75% ETA n-6 EE relative to the total omega-3 fatty acid ethyl esters. In some embodiments, the oral composition comprises less than 1.5% ETA n-6 EE relative to the total omega-3 fatty acid ethyl esters. In some embodiments, the oral composition comprises less than 1.25% ETA n-6 EE relative to the total omega-3 fatty acid ethyl esters. In some embodiments, the oral composition comprises less than 1% ETA n-6 EE relative to the total omega-3 fatty acid ethyl esters. In some embodiments, the oral composition comprises less than 0.9% ETA n-6 EE relative to the total omega-3 fatty acid ethyl esters. In some embodiments, the oral composition comprises less than 0.8% ETA n-6 EE relative to the total omega-3 fatty acid ethyl esters. In some embodiments, the oral composition comprises less than 0.75% ETA n-6 EE relative to the total omega-3 fatty acid ethyl esters. In some embodiments, the oral composition comprises less than 0.5% ETA n-6 EE relative to the total omega-3 fatty acid ethyl esters. In some embodiments, the oral composition comprises less than 0.25% ETA n-6 EE relative to the total omega-3 fatty acid ethyl esters.

In some embodiments, the oral composition of the present disclosure (e.g., oral pharmaceutical composition or dietary supplement) comprises less than 6% docosapentaenoic acid ethyl ester (DPA n-6 EE) relative to the total omega-3 fatty acid ethyl esters. In some embodiments, the oral composition comprises less than 5% DPA n-6 EE relative to the total omega-3 fatty acid ethyl esters. In some embodiments, the oral composition comprises less than 4% DPA n-6 EE relative to the total omega-3 fatty acid ethyl esters. In some embodiments, the oral composition comprises less than 3% DPA n-6 EE relative to the total omega-3 fatty acid ethyl esters. In some embodiments, the oral composition comprises less than 2.5% docosapentaenoic acid ethyl ester (DPA n-6 EE) relative to the total omega-3 fatty acid ethyl esters, such as less than 1%. In some embodiments, the oral composition comprises less than 2.25% DPA n-6 EE relative to the total omega-3 fatty acid ethyl esters. In some embodiments, the oral composition comprises less than 2% DPA n-6 EE relative to the total omega-3 fatty acid ethyl esters. In some embodiments, the oral composition comprises less than 1.75% DPA n-6 EE relative to the total omega-3 fatty acid ethyl esters. In some embodiments, the oral composition comprises less than 1.5% DPA n-6 EE relative to the total omega-3 fatty acid ethyl esters. In some embodiments, the oral composition comprises less than 1.25% DPA n-6 EE relative to the total omega-3 fatty acid ethyl esters. In some embodiments, the oral composition comprises less than 1% DPA n-6 EE relative to the total omega-3 fatty acid ethyl esters. In some embodiments, the oral composition comprises less than 0.9% DPA n-6 EE relative to the total omega-3 fatty acid ethyl esters. In some embodiments, the oral composition comprises less than 0.8% DPA n-6 EE relative to the total omega-3 fatty acid ethyl esters. In some embodiments, the oral composition comprises less than 0.75% DPA n-6 EE relative to the total omega-3 fatty acid ethyl esters. In some embodiments, the oral composition comprises less than 0.7% DPA n-6 EE relative to the total omega-3 fatty acid ethyl esters. In some embodiments, the oral composition comprises less than 0.6% DPA n-6 EE relative to the total omega-3 fatty acid ethyl esters. In some embodiments, the oral composition comprises less than 0.5% DPA n-6 EE relative to the total omega-3 fatty acid ethyl esters. In some embodiments, the oral composition comprises less than 0.25% DPA n-6 EE relative to the omega-3 fatty acid ethyl esters.

In some embodiments, provided herein is an oral composition comprising a mixture of fatty acid esters comprising omega-3 fatty acid ethyl esters and omega-6 fatty acid ethyl esters, wherein the pharmaceutical composition comprises less than 2% omega-6 fatty acid ethyl esters relative to the omega-3 fatty acid ethyl esters and a liquid carrier.

### Liquid Carriers

The oral composition of the present disclosures (e.g., pharmaceutical composition or dietary supplement) comprises a liquid carrier to serve, among other things, as a solvent to other components of the oral composition. In some embodiments, the liquid carrier comprises an edible oil derived from a non-animal source. In some embodiments, the liquid carrier is non-aqueous, i.e., comprises less than 2 wt % water, such as less than 1 wt % water, less than 0.5 wt % water, less than 0.2 wt % water, or less than 0.1 wt % water.

In some embodiments, the liquid carrier comprises olive oil. In some embodiments, the olive oil is extra virgin, virgin olive oil, refined olive oil (e.g., grade IV), extra light olive oil, light olive oil, or flavored olive oil. In some embodiments, the olive oil is refined (grade IV) olive oil.

In some embodiments, the liquid carrier further comprises one or more liquids (e.g., non-aqueous liquids) other than olive oil. In some embodiments, the liquid is selected from safflower oil, high-oleic safflower oil, sunflower oil, high-oleic sunflower oil, rapeseed oil, almond oil, avocado oil, sesame oil, soybean oil, corn oil, peanut oil, palm oil, cottonseed oil, flaxseed oil, coconut oil, grapeseed oil, hempseed oil, oleinate (e.g., a monounsaturated fatty acid), canola oil, linseed oil, walnut oil, wheat germ oil, pumpkin seed oil, rice bran oil, and combinations thereof.

In some embodiments, the non-aqueous liquid is safflower oil. In some embodiments, the non-aqueous liquid is high-oleic safflower oil. In some embodiments, the non-aqueous liquid is sunflower oil. In some embodiments, the non-aqueous liquid is high-oleic sunflower oil. In some embodiments, the non-aqueous liquid is rapeseed oil. In some embodiments, the non-aqueous liquid is almond oil. In some embodiments, the non-aqueous liquid is avocado oil. In some embodiments, the non-aqueous liquid is sesame oil. In some embodiments, the non-aqueous liquid is soybean oil. In some embodiments, the non-aqueous liquid is corn oil. In some embodiments, the non-aqueous liquid is peanut oil. In some embodiments, the non-aqueous liquid is palm oil. In some embodiments, the non-aqueous liquid is cottonseed oil. In some embodiments, the non-aqueous liquid is flaxseed oil. In some embodiments, the non-aqueous liquid is coconut oil. In some embodiments, the non-aqueous liquid is grapeseed oil. In some embodiments, the non-aqueous liquid is hempseed oil. In some embodiments, the non-aqueous liquid is oleinate (e.g., a polyunsaturated fatty acid). In some embodiments, the non-aqueous liquid is canola oil. In some embodiments, the non-aqueous liquid is linseed oil. In some embodiments, the non-aqueous liquid is walnut oil. In some embodiments, the non-aqueous liquid is wheat germ oil. In some embodiments, the non-aqueous liquid is pumpkin seed oil. In some embodiments, the non-aqueous liquid is rice bran oil.

In some embodiments, the liquid carrier is refined olive oil (e.g., grade IV). In some embodiments, the refined olive oil was produced by silica chromatography. In some embodiments, the refined olive oil meets any one of or more characteristics, such as acid value, peroxide value, water content, unsaponifiable matter, alkaline impurities, sesame oil, specific absorbance, fatty acid composition, saturated fatty acids, palmitic acid, palmitoleic acid, stearicstearic acid, oleic acid (and isomer(s)), linoleic acid, linolenic acid, arachidic acid, eicosenoic acid, behenic acid, lignoceric acid, sterol composition, cholesterol, campesterol, 47-stigmasterol, or sum of β-sitosterol, Δ5,23-stigmastadienol, clerosterol, sitostanol, 45-avenasterol, and Δ5,24-stigmastadienol.

### Olive Oil Acid Value

In some embodiments, the refined olive oil is characterized by an acid value (mg KOH/g) of 0.5 or less, e.g., 0.45, 0.4, 0.35, 0.3, 0.25, 0.2, 0.15, 0.1, or 0.05 or less. In some embodiments, the refined olive oil is characterized by an acid value of 0.5 or less. In some embodiments, the refined olive oil is characterized by an acid value of 0.45 or less. In some embodiments, the refined olive oil is characterized by an acid value of 0.4 or less. In some embodiments, the refined olive oil is characterized by an acid value of 0.35 or less. In some embodiments, the refined olive oil is characterized by an acid value of 0.3 or less. In some embodiments, the refined olive oil is characterized by an acid value of 0.25 or less. In some embodiments, the refined olive oil is characterized by an acid value of 0.2 or less. In some embodiments, the refined olive oil is characterized by an acid value of 0.15 or less. In some embodiments, the refined olive oil is characterized by an acid value of 0.1 or less. In some embodiments, the refined olive oil is characterized by an acid value of 0.05 or less.

### Peroxide Value

In some embodiments, the refined olive oil is characterized by a peroxide value (mEq O₂/kg) of 7.5 or less, e.g., 7, 6.5, 6, 5, 4.5, 4, 3.5, 3, 2.5, 2, 1.5, 1, 0.5, or 0.1 or less. In some embodiments, the refined olive oil is characterized by a peroxide value of 7.5 or less. In some embodiments, the refined olive oil is characterized by a peroxide value of 7 or less. In some embodiments, the refined olive oil is characterized by a peroxide value of 6.5 or less. In some embodiments, the refined olive oil is characterized by a peroxide value of 6 or less. In some embodiments, the refined olive oil is characterized by a peroxide value of 5.5 or less. In some embodiments, the refined olive oil is characterized by a peroxide value of 5 or less. In some embodiments, the refined olive oil is characterized by a peroxide value of 4.5 or less. In some embodiments, the refined olive oil is characterized by a peroxide value of 4 or less. In some embodiments, the refined olive oil is characterized by a peroxide value of 3.5 or less. In some embodiments, the refined olive oil is characterized by a peroxide value of 3 or less. In some embodiments, the refined olive oil is characterized by a peroxide value of 2.5 or less. In some embodiments, the refined olive oil is characterized by a peroxide value of 2 or less. In some embodiments, the refined olive oil is characterized by a peroxide value of 1.5 or less. In some embodiments, the refined olive oil is characterized by a peroxide value of 1 or less. In some embodiments, the refined olive oil is characterized by a peroxide value of 0.5 or less. In some embodiments, the refined olive oil is characterized by a peroxide value of 0.1 or less.

### Unsaponifiable Matter (%)

In some embodiments, the refined olive oil comprises an unsaponifiable matter percentage less than 1.5%. In some embodiments, the unsaponifiable matter is less than 5%, 4.5%, 4%, 3.5%, 3%, 2.75%, 2.5%, 2.25%, 2%, 1.75%, 1.5%, 1.4%, 1.3%, 1.25%, 1.15%, 1%, 0.9%, 0.75%, 0.65%, 0.5%, 0.35%, 0.25%, 0.15%, 0.1%, 0.05%, or 0.01%. In some embodiments, the unsaponifiable matter is less than 1.5%. In some embodiments, the unsaponifiable matter is less than 1.4%. In some embodiments, the unsaponifiable matter is less than 1.3%. In some embodiments, the unsaponifiable matter is less than 1.25%. In some embodiments, the unsaponifiable matter is less than 1.15%. In some embodiments, the unsaponifiable matter is less than 1%. In some embodiments, the unsaponifiable matter is less than 0.9%. In some embodiments, the unsaponifiable matter is less than 0.75%. In some embodiments, the unsaponifiable matter is less than 0.65%. In some embodiments, the unsaponifiable matter is less than 0.5%. In some embodiments, the unsaponifiable matter is less than 0.35%. In some embodiments, the unsaponifiable matter is less than 0.25%. In some embodiments, the unsaponifiable matter is less than 0.15%. In some embodiments, the unsaponifiable matter is less than 0.1%. In some embodiments, the unsaponifiable matter is less than 0.05%. In some embodiments, the unsaponifiable matter is less than 0.01%.

### Specific Absorbance

In some embodiments, the refined olive oil is characterized by specific absorbance at 270 nm of 1.25 or less, e.g., 1.20, 1.15, 1.10, 1.05, 1.00, 0.95, 0.90, 0.85, 0.80, 0.75, 0.70, 0.65, 0.60, 0.55, 0.50, 0.45, 0.40, 0.35, 0.30, 0.25, 0.20, 0.15, 0.10, 0.05, or 0.01 or less. In some embodiments, the refined olive oil is characterized by specific absorbance at 270 nm of 1.25 or less. In some embodiments, the refined olive oil is characterized by specific absorbance at 270 nm of 1.20 or less. In some embodiments, the refined olive oil is characterized by specific absorbance at 270 nm of 1.15 or less. In some embodiments, the refined olive oil is characterized by specific absorbance at 270 nm of 1.10 or less. In some embodiments, the refined olive oil is characterized by specific absorbance at 270 nm of 1.05 or less. In some embodiments, the refined olive oil is characterized by specific absorbance at 270 nm of 1.00 or less. In some embodiments, the refined olive oil is characterized by specific absorbance at 270 nm of 0.95 or less. In some embodiments, the refined olive oil is characterized by specific absorbance at 270 nm of 0.90 or less. In some embodiments, the refined olive oil is characterized by specific absorbance at 270 nm of 0.85 or less. In some embodiments, the refined olive oil is characterized by specific absorbance at 270 nm of 0.80 or less. In some embodiments, the refined olive oil is characterized by specific absorbance at 270 nm of 0.75 or less. In some embodiments, the refined olive oil is characterized by specific absorbance at 270 nm of 0.70 or less. In some embodiments, the refined olive oil is characterized by specific absorbance at 270 nm of 0.65 or less. In some embodiments, the refined olive oil is characterized by specific absorbance at 270 nm 0.60 or less. In some embodiments, the refined olive oil is characterized by specific absorbance at 270 nm of 0.55 or less. In some embodiments, the refined olive oil is characterized by specific absorbance at 270 nm of 0.50 or less. In some embodiments, the refined olive oil is characterized by specific absorbance at 270 nm of 0.45 or less. In some embodiments, the refined olive oil is characterized by specific absorbance at 270 nm of 0.40 or less. In some embodiments, the refined olive oil is characterized by specific absorbance at 270 nm of 0.35 or less. In some embodiments, the refined olive oil is characterized by specific absorbance at 270 nm of 0.30 or less. In some embodiments, the refined olive oil is characterized by specific absorbance at 270 nm of 0.25 or less. In some embodiments, the refined olive oil is characterized by specific absorbance at 270 nm of 0.20 or less. In some embodiments, the refined olive oil is characterized by specific absorbance at 270 nm of 0.15 or less. In some embodiments, the refined olive oil is characterized by specific absorbance at 270 nm of 0.10 or less. In some embodiments, the refined olive oil is characterized by specific absorbance at 270 nm of 0.05 or less. In some embodiments, the refined olive oil is characterized by specific absorbance at 270 nm of 0.01 or less.

### Saturated Fatty Acids

In some embodiments, the refined olive oil comprises less than 0.2% saturated fatty acids. In some embodiments, the refined olive oil comprises less than 0.15% saturated fatty acids. In some embodiments, the refined olive oil comprises less than 0.1% saturated fatty acids. In some embodiments, the refined olive oil comprises less than 0.05% saturated fatty acids. In some embodiments, the refined olive oil comprises less than 0.025% saturated fatty acids. In some embodiments, the refined olive oil comprises less than 0.01% saturated fatty acids.

### Palmitic Acid

In some embodiments, the refined olive oil fatty acid fraction comprises from 5% to 25% palmitic acid by weight. In some embodiments, the refined olive oil fatty acid fraction comprises from 5.5% to 24% palmitic acid. In some embodiments, the refined olive oil fatty acid fraction comprises from 6% to 23% palmitic acid. In some embodiments, the refined olive oil fatty acid fraction comprises from 6.5% to 22% palmitic acid. In some embodiments, the refined olive oil fatty acid fraction comprises from 7% to 21% palmitic acid. In some embodiments, the refined olive oil fatty acid fraction comprises from 7.5% to 20% palmitic acid. In some embodiments, the refined olive oil fatty acid fraction comprises from 8% to 19% palmitic acid. In some embodiments, the refined olive oil fatty acid fraction comprises from 8.5% to 18% palmitic acid. In some embodiments, the refined olive oil fatty acid fraction comprises from 9% to 17% palmitic acid. In some embodiments, the refined olive oil fatty acid fraction comprises from 9.5% to 16% palmitic acid. In some embodiments, the refined olive oil fatty acid fraction comprises from 9.5% to 15% palmitic acid. In some embodiments, the refined olive oil fatty acid fraction comprises from 9.5% to 12% palmitic acid. In some embodiments, the refined olive oil fatty acid fraction comprises from 9.5% to 11% palmitic acid.

### Palmitoleic Acid

In some embodiments, the refined olive oil fatty acid fraction comprises less than 6%, 5.5%, 5%, 4.5%, 4%, 3.5%, 3%, 2.5%, 2%, 1.5%, 1%, 0.5%, or 0.25% palmitoleic acid by weight. In some embodiments, the refined olive oil fatty acid fraction comprises less than 3.5% palmitoleic acid. In some embodiments, the refined olive oil fatty acid fraction comprises less than 3% palmitoleic acid. In some embodiments, the refined olive oil fatty acid fraction comprises less than 2.5% palmitoleic acid. In some embodiments, the refined olive oil fatty acid fraction comprises less than 2% palmitoleic acid. In some embodiments, the refined olive oil fatty acid fraction comprises less than 1.5% palmitoleic acid. In some embodiments, the refined olive oil fatty acid fraction comprises less than 1% palmitoleic acid. In some embodiments, the refined olive oil fatty acid fraction comprises less than 0.5% palmitoleic acid. In some embodiments, the refined olive oil fatty acid fraction comprises less than 0.25% palmitoleic acid.

### Stearic Acid

In some embodiments, the refined olive oil fatty acid fraction comprises stearic acid in an amount from 0.01% to 10%. In some embodiments, the refined olive oil fatty acid fraction comprises stearic acid in an amount from 0.1% to 7%. In some embodiments, the refined olive oil fatty acid fraction comprises stearic acid in an amount from 0.25% to 6%. In some embodiments, the refined olive oil fatty acid fraction comprises stearic acid in an amount from 0.5% to 6% by weight. In some embodiments, the refined olive oil fatty acid fraction comprises stearic acid in an amount from 0.5% to 5%. In some embodiments, the refined olive oil fatty acid fraction comprises stearic acid in an amount from 0.5% to 4%. In some embodiments, the refined olive oil fatty acid fraction comprises stearic acid in an amount from 0.5% to 3%. In some embodiments, the refined olive oil fatty acid fraction comprises stearic acid in an amount from 1% to 6%. In some embodiments, the refined olive oil fatty acid fraction comprises stearic acid in an amount from 1.5% to 6%. In some embodiments, the refined olive oil fatty acid fraction comprises stearic acid in an amount from 2% to 6%. In some embodiments, the refined olive oil fatty acid fraction comprises stearic acid in an amount from 3.5% to 6%. In some embodiments, the refined olive oil fatty acid fraction comprises stearic acid in an amount from 3.5% to 5%. In some embodiments, the refined olive oil fatty acid fraction comprises stearic acid in an amount from 1.5% to 4%. In some embodiments, the refined olive oil fatty acid fraction comprises stearic acid in an amount from 2.5% to 4%. In some embodiments, the refined olive oil fatty acid fraction comprises stearic acid in an amount from 3% to 4%.

### Oleic Acid

In some embodiments, the refined olive oil fatty acid fraction comprises from 55% to 85% oleic acid (e.g., with isomers) by weight. In some embodiments, the refined olive oil fatty acid fraction comprises from 60% to 85% oleic acid (e.g., with isomers). In some embodiments, the refined olive oil fatty acid fraction comprises from 65% to 85% oleic acid (e.g., with isomers). In some embodiments, the refined olive oil fatty acid fraction comprises from 75% to 85% oleic acid (e.g., with isomers). In some embodiments, the refined olive oil fatty acid fraction comprises from 80% to 85% oleic acid (e.g., with isomers). In some embodiments, the refined olive oil fatty acid fraction comprises from 75% to 81% oleic acid (e.g., with isomers).

### Linoleic Acid

In some embodiments, the refined olive oil fatty acid fraction comprises from 3% to 20% linoleic acid by weight. In some embodiments, the refined olive oil fatty acid fraction comprises from 3% to 18% linoleic acid. In some embodiments, the refined olive oil fatty acid fraction comprises from 3% to 16% linoleic acid. In some embodiments, the refined olive oil fatty acid fraction comprises from 3% to 14% linoleic acid. In some embodiments, the refined olive oil fatty acid fraction comprises from 3% to 12% linoleic acid. In some embodiments, the refined olive oil fatty acid fraction comprises from 3% to 10% linoleic acid. In some embodiments, the refined olive oil fatty acid fraction comprises from 3% to 8% linoleic acid. In some embodiments, the refined olive oil fatty acid fraction comprises from 3% to 6% linoleic acid. In some embodiments, the refined olive oil fatty acid fraction comprises from 3.5% to 5% linoleic acid. In some embodiments, the refined olive oil fatty acid fraction comprises from 4% to 5% linoleic acid. In some embodiments, the refined olive oil fatty acid fraction comprises from 4.5% to 5% linoleic acid.

### Linolenic Acid

In some embodiments, the refined olive oil fatty acid fraction comprises 1.5% or less linolenic acid by weight, e.g., 1.2%, 1.1%, 1%, 0.9%, 0.8%, 0.7%, 0.6%, 0.5%, 0.4%, 0.3%, 0.2%, or 0.1% or less linolenic acid by weight. In some embodiments, the refined olive oil fatty acid fraction comprises 1.5% or less linolenic acid. In some embodiments, the refined olive oil fatty acid fraction comprises 1.2% or less linolenic acid. In some embodiments, the refined olive oil fatty acid fraction comprises 1.1% or less linolenic acid. In some embodiments, the refined olive oil fatty acid fraction comprises 1% or less linolenic acid. In some embodiments, the refined olive oil fatty acid fraction comprises 0.9% or less linolenic acid. In some embodiments, the refined olive oil fatty acid fraction comprises 0.8% or less linolenic acid. In some embodiments, the refined olive oil fatty acid fraction comprises 0.7% or less linolenic acid. In some embodiments, the refined olive oil fatty acid fraction comprises 0.6% or less linolenic acid. In some embodiments, the refined olive oil fatty acid fraction comprises 0.5% or less linolenic acid. In some embodiments, the refined olive oil fatty acid fraction comprises 0.4% or less linolenic acid. In some embodiments, the refined olive oil fatty acid fraction comprises 0.3% or less linolenic acid. In some embodiments, the refined olive oil fatty acid fraction comprises 0.2% or less linolenic acid. In some embodiments, the refined olive oil fatty acid fraction comprises 0.1% or less linolenic acid.

### Arachidic Acid

In some embodiments, the refined olive oil fatty acid fraction comprises 0.8% arachidic acid by weight, e.g., 0.7%, 0.6%, 0.5%, 0.4%, 0.3%, 0.2%, or 0.1% or less arachidic acid by weight. In some embodiments, the refined olive oil fatty acid fraction comprises 0.8% or less arachidic acid. In some embodiments, the refined olive oil fatty acid fraction comprises 0.7% or less arachidic acid. In some embodiments, the refined olive oil fatty acid fraction comprises 0.6% or less arachidic acid. In some embodiments, the refined olive oil fatty acid fraction comprises 0.5% or less arachidic acid. In some embodiments, the refined olive oil fatty acid fraction comprises 0.4% or less arachidic acid. In some embodiments, the refined olive oil fatty acid fraction comprises 0.3% or less arachidic acid. In some embodiments, the refined olive oil fatty acid fraction comprises 0.2% or less arachidic acid. In some embodiments, the refined olive oil fatty acid fraction comprises 0.1% or less arachidic acid.

### Eicosenoic Acid

In some embodiments, the refined olive oil fatty acid fraction comprises 0.5% or less eicosenoic acid by weight, e.g., 0.4%, 0.3%, 0.2%, or 0.1% or less eicosenoic acid by weight. In some embodiments, the refined olive oil fatty acid fraction comprises 0.5% or less eicosenoic acid. In some embodiments, the refined olive oil fatty acid fraction comprises 0.4% or less eicosenoic acid. In some embodiments, the refined olive oil fatty acid fraction comprises 0.3% or less eicosenoic acid. In some embodiments, the refined olive oil fatty acid fraction comprises 0.2% or less eicosenoic acid. In some embodiments, the refined olive oil fatty acid fraction comprises 0.1% or less eicosenoic acid.

### Behenic Acid

In some embodiments, the refined olive oil fatty acid fraction comprises 0.5% or less behenic acid by weight, e.g., 0.4%, 0.3%, 0.2%, or 0.1% or less behenic acid by weight. In some embodiments, the refined olive oil fatty acid fraction comprises 0.5% or less behenic acid. In some embodiments, the refined olive oil fatty acid fraction comprises 0.4% or less behenic acid. In some embodiments, the refined olive oil fatty acid fraction comprises 0.3% or less behenic acid. In some embodiments, the refined olive oil fatty acid fraction comprises 0.2% or less behenic acid. In some embodiments, the refined olive oil fatty acid fraction comprises 0.1% or less behenic acid.

### Lignoceric Acid

In some embodiments, the refined olive oil fatty acid fraction comprises 0.5% or less lignoceric acid by weight, e.g., 0.4%, 0.3%, 0.2%, or 0.1% or less lignoceric acid by weight. In some embodiments, the refined olive oil fatty acid fraction comprises 0.5% or less lignoceric acid. In some embodiments, the refined olive oil fatty acid fraction comprises 0.4% or less lignoceric acid. In some embodiments, the refined olive oil fatty acid fraction comprises 0.3% or less lignoceric acid. In some embodiments, the refined olive oil fatty acid fraction comprises 0.2% or less lignoceric acid. In some embodiments, the refined olive oil fatty acid fraction comprises 0.1% or less lignoceric acid.

### Campesterol

In some embodiments, the refined olive oil sterol fraction comprises 5% or less campesterol by weight. In some embodiments, the refined olive oil sterol fraction comprises 4% or less campesterol. In some embodiments, the refined olive oil sterol fraction comprises 3% or less campesterol. In some embodiments, the refined olive oil sterol fraction comprises 2% or less campesterol. In some embodiments, the refined olive oil sterol fraction comprises 1% or less campesterol.

### Δ7-Stigmasterol

In some embodiments, the refined olive oil sterol fraction comprises less than 0.5%, 0.4%, 0.3%, 0.2%, or 0.1% Δ7-stigmasterol by weight. In some embodiments, the refined olive oil sterol fraction comprises less than 0.5% Δ7-stigmasterol. In some embodiments, the refined olive oil sterol fraction comprises less than 0.4% Δ7-stigmasterol. In some embodiments, the refined olive oil sterol fraction comprises less than 0.3% Δ7-stigmasterol. In some embodiments, the refined olive oil sterol fraction comprises less than 0.2% Δ7-stigmasterol. In some embodiments, the refined olive oil sterol fraction comprises less than 0.1% Δ7-stigmasterol.

Sum of β-sitosterol, Δ5,23-stigmastadiencol, clerosterol, sitostanol, Δ5-avenasterol, and Δ-5,24-stigmastadienol

In some embodiments, the refined olive oil sterol fraction comprises a sum of β-sitosterol, Δ5,23-stigmastadienol, clerosterol, sitostanol, Δ5-avenasterol, and Δ5,24-stigmastadienol of greater than 90%. In some embodiments, the refined olive oil sterol fraction comprises a sum of β-sitosterol, Δ5,23-stigmastadiencol, clerosterol, sitostanol, Δ5-avenasterol, and Δ-5,24-stigmastadienol of greater than 91%. In some embodiments, the refined olive oil sterol fraction comprises a sum of β-sitosterol, Δ5,23-stigmastadiencol, clerosterol, sitostanol, Δ5-avenasterol, and Δ-5,24-stigmastadienol of greater than 92%. In some embodiments, the olive oil sterol fraction comprises a sum of β-sitosterol, Δ5,23-stigmastadiencol, clerosterol, sitostanol, Δ5-avenasterol, and Δ-5,24-stigmastadienol of greater than 93%. In some embodiments, the refined olive oil sterol fraction comprises a sum of β-sitosterol, Δ5,23-stigmastadiencol, clerosterol, sitostanol, Δ5-avenasterol, and Δ-5,24-stigmastadienol of greater than 94%. In some embodiments, the refined olive oil sterol fraction comprises a sum of β-sitosterol, Δ5,23-stigmastadiencol, clerosterol, sitostanol, Δ5-avenasterol, and Δ-5,24-stigmastadienol of greater than 95%. In some embodiments, the refined olive oil sterol fraction comprises a sum of β-sitosterol, Δ5,23-stigmastadiencol, clerosterol, sitostanol, Δ5-avenasterol, and Δ-5,24-stigmastadienol of greater than 96%. In some embodiments, the refined olive oil sterol fraction comprises a sum of β-sitosterol, Δ5,23-stigmastadiencol, clerosterol, sitostanol, Δ5-avenasterol, and Δ-5,24-stigmastadienol of greater than 97%. In some embodiments, the refined olive oil sterol fraction comprises a sum of β-sitosterol, Δ5,23-stigmastadiencol, clerosterol, sitostanol, Δ5-avenasterol, and Δ-5,24-stigmastadienol of greater than 98%. In some embodiments, the refined olive oil sterol fraction comprises a sum of β-sitosterol, Δ5,23-stigmastadiencol, clerosterol, sitostanol, Δ5-avenasterol, and Δ-5,24-stigmastadienol of greater than 99%.

In some embodiments, the refined olive oil comprises less stigmasterol than campesterol.

### Antioxidants

In some embodiments, the oral composition of the present disclosure (e.g., oral pharmaceutical composition or dietary supplement) may contain at least one antioxidant.

In some embodiments, the antioxidant is selected from ascorbyl palmitate (vitamin C), lecithin, alpha-tocopherol (vitamin E), vitamin A (carotenoids, retinoids), vitamin D2 (ergocalciferol), vitamin E (tocopherols), chitin, chitosan, quercetin, ubiquinol, ubiquinone (Coenzyme Q), α-carotene, β-carotene, lycopene, lutein, zeaxanthin, and β-cryptoxanthin β-carote, astaxanthin, bixin, norbixin, capsanthin, canthaxanthin, β-Apo-8-carotenal, and β-apo-8-carotenal-ester, myricetin, isoflavone phytoestrogens, resveratrol, pterostilbene, chicoric acid, cinnamic acid and its derivatives, ellagic acid, ellagitannins, gallic acid, rosmarinic acid, salicylic acid, curcumin, silymarin, anthocyanins, caffeic acid, vanillin, tannins, ferulic acid, flavonoids, phenolic acids, stilbenes, lignans, catechins, ascorbic acid, allicin, plant essential oils (e.g., oregano, thyme, lemon, lavender, peppermint, tea tree, cedarwood, eucalyptus, lemongrass, cinnamon bark, rosemary, geranium, citronella, clove, mint, fennel, coriander, myrrh, marjoram), and combinations thereof.

In some embodiments, the antioxidant is selected from ascorbyl palmitate, lecithin, and alpha-tocopherol. In some embodiments, the antioxidant is a mixture of ascorbyl palmitate, lecithin, and alpha-tocopherol. In some embodiments, the antioxidant is a mixture of ascorbyl palmitate and lecithin. In some embodiments, the antioxidant is a mixture of ascorbyl palmitate and alpha-tocopherol. In some embodiments, the antioxidant is a mixture of lecithin and alpha-tocopherol.

In some embodiments, the antioxidant is ascorbyl palmitate. In some embodiments, the antioxidant is lecithin. In some embodiments, the antioxidant is alpha-tocopherol. In some embodiments, the antioxidant is vitamin A (carotenoids, retinoids). In some embodiments, the antioxidant is vitamin D2 (ergocalciferol). In some embodiments, the antioxidant is vitamin E (tocopherols). In some embodiments, the antioxidant is chitin. In some embodiments, the antioxidant is chitosan. In some embodiments, the antioxidant is quercetin. In some embodiments, the antioxidant is ubiquinol. In some embodiments, the antioxidant is ubiquinone (Coenzyme Q). In some embodiments, the antioxidant is α-carotene. In some embodiments, the antioxidant is β-carotene. In some embodiments, the antioxidant is lycopene. In some embodiments, the antioxidant is lutein. In some embodiments, the antioxidant is zeaxanthin. In some embodiments, the antioxidant is β-cryptoxanthin β-carotene. In some embodiments, the antioxidant is astaxanthin. In some embodiments, the antioxidant is bixin. In some embodiments, the antioxidant is norbixin. In some embodiments, the antioxidant is capsanthin. In some embodiments, the antioxidant is canthaxanthin. In some embodiments, the antioxidant is β-Apo-8-carotenal. In some embodiments, the antioxidant is β-apo-8-carotenal-ester. In some embodiments, the antioxidant is myricetin. In some embodiments, the antioxidant is isoflavone phytoestrogens. In some embodiments, the antioxidant is resveratrol. In some embodiments, the antioxidant is pterostilbene. In some embodiments, the antioxidant is chicoric acid. In some embodiments, the antioxidant is cinnamic acid and its derivatives. In some embodiments, the antioxidant is ellagic acid. In some embodiments, the antioxidant is ellagitannins. In some embodiments, the antioxidant is gallic acid. In some embodiments, the antioxidant is rosmarinic acid. In some embodiments, the antioxidant is salicylic acid. In some embodiments, the antioxidant is curcumin. In some embodiments, the antioxidant is silymarin. In some embodiments, the antioxidant is anthocyanins. In some embodiments, the antioxidant is caffeic acid.

In some embodiments, the at least one antioxidant is present in the oral composition of the present disclosure in an amount from 0.1 mg/10 mL to 250 mg/10 mL, such as from 35 mg/10 mL to 200 mg/10 mL, from 35 mg/10 mL to 200 mg/10 mL, from 35 mg/10 mL to 150 mg/10 mL, from 35 mg/10 mL to 100 mg/10 mL, or from 35 mg/10 mL to 75 mg/10 mL. In some embodiments, the at least one antioxidant is present in the oral composition in an amount from 0.1 mg/10 mL to 240 mg/10 mL. In some embodiments, the at least one antioxidant is present in the oral composition in an amount from 0.1 mg/10 mL to 230 mg/10 mL. In some embodiments, the at least one antioxidant is present in the oral composition in an amount from 0.1 mg/10 mL to 220 mg/10 mL. In some embodiments, the at least one antioxidant is present in the oral composition in an amount from 0.1 mg/10 mL to 210 mg/10 mL. In some embodiments, the at least one antioxidant is present in the oral composition in an amount from 0.1 mg/10 mL to 200 mg/10 mL. In some embodiments, the at least one antioxidant is present in the oral composition in an amount from 0.1 mg/10 mL to 190 mg/10 mL. In some embodiments, the at least one antioxidant is present in the oral composition in an amount from 0.1 mg/10 mL to 180 mg/10 mL. In some embodiments, the at least one antioxidant is present in the oral composition in an amount from 0.1 mg/10 mL to 170 mg/10 mL. In some embodiments, the at least one antioxidant is present in the oral composition in an amount from 0.1 mg/10 mL to 160 mg/10 mL. In some embodiments, the at least one antioxidant is present in the oral composition in an amount from 0.1 mg/10 mL to 150 mg/10 mL. In some embodiments, the at least one antioxidant is present in the oral composition in an amount from 0.1 mg/10 mL to 140 mg/10 mL. In some embodiments, the at least one antioxidant is present in the oral composition in an amount from 0.1 mg/10 mL to 130 mg/10 mL. In some embodiments, the at least one antioxidant is present in the oral composition in an amount from 0.1 mg/10 mL to 120 mg/10 mL. In some embodiments, the at least one antioxidant is present in the oral composition in an amount from 0.1 mg/10 mL to 110 mg/10 mL. In some embodiments, the at least one antioxidant is present in the oral composition in an amount from 0.1 mg/10 mL to 100 mg/10 mL. In some embodiments, the at least one antioxidant is present in the oral composition in an amount from 0.1 mg/10 mL to 90 mg/10 mL. In some embodiments, the at least one antioxidant is present in the oral composition in an amount from 0.1 mg/10 mL to 80 mg/10 mL. In some embodiments, the at least one antioxidant is present in the oral composition in an amount from 0.1 mg/10 mL to 70 mg/10 mL. In some embodiments, the at least one antioxidant is present in the oral composition in an amount from 0.1 mg/10 mL to 60 mg/10 mL. In some embodiments, the at least one antioxidant is present in the oral composition in an amount from 0.1 mg/10 mL to 50 mg/10 mL. In some embodiments, the at least one antioxidant is present in the oral composition in an amount from 0.1 mg/10 mL to 40 mg/10 mL. In some embodiments, the at least one antioxidant is present in the oral composition in an amount from 0.1 mg/10 mL to 30 mg/10 mL. In some embodiments, the at least one antioxidant is present in the oral composition in an amount from 0.1 mg/10 mL to 20 mg/10 mL. In some embodiments, the at least one antioxidant is present in the oral composition in an amount from 0.1 mg/10 mL to 10 mg/10 mL. In some embodiments, the at least one antioxidant is present in the oral composition in an amount from 0.1 mg/10 mL to 5 mg/10 mL. In some embodiments, the at least one antioxidant is present in the oral composition in an amount from 0.1 mg/10 mL to 1 mg/10 mL.

In some embodiments, the at least one antioxidant is present in the oral composition in an amount from 1 mg/10 mL to 250 mg/10 mL. In some embodiments, the at least one antioxidant is present in the oral composition in an amount from 5 mg/10 mL to 250 mg/10 mL. In some embodiments, the at least one antioxidant is present in the oral composition in an amount from 10 mg/10 mL to 250 mg/10 mL. In some embodiments, the at least one antioxidant is present in the oral composition in an amount from 20 mg/10 mL to 250 mg/10 mL. In some embodiments, the at least one antioxidant is present in the oral composition in an amount from 30 mg/10 mL to 250 mg/10 mL. In some embodiments, the at least one antioxidant is present in the oral composition in an amount from 40 mg/10 mL to 250 mg/10 mL. In some embodiments, the at least one antioxidant is present in the oral composition in an amount from 50 mg/10 mL to 250 mg/10 mL. In some embodiments, the at least one antioxidant is present in the oral composition in an amount from 60 mg/10 mL to 250 mg/10 mL. In some embodiments, the at least one antioxidant is present in the oral composition in an amount from 70 mg/10 mL to 250 mg/10 mL. In some embodiments, the at least one antioxidant is present in the oral composition in an amount from 80 mg/10 mL to 250 mg/10 mL. In some embodiments, the at least one antioxidant is present in the oral composition in an amount from 90 mg/10 mL to 250 mg/10 mL. In some embodiments, the at least one antioxidant is present in the oral composition in an amount from 100 mg/10 mL to 250 mg/10 mL. In some embodiments, the at least one antioxidant is present in the oral composition in an amount from 110 mg/10 mL to 250 mg/10 mL. In some embodiments, the at least one antioxidant is present in the oral composition in an amount from 120 mg/10 mL to 250 mg/10 mL. In some embodiments, the at least one antioxidant is present in the oral composition in an amount from 130 mg/10 mL to 250 mg/10 mL. In some embodiments, the at least one antioxidant is present in the oral composition in an amount from 140 mg/10 mL to 250 mg/10 mL. In some embodiments, the at least one antioxidant is present in the oral composition in an amount from 150 mg/10 mL to 250 mg/10 mL. In some embodiments, the at least one antioxidant is present in the oral composition in an amount from 160 mg/10 mL to 250 mg/10 mL. In some embodiments, the at least one antioxidant is present in the oral composition in an amount from 170 mg/10 mL to 250 mg/10 mL. In some embodiments, the at least one antioxidant is present in the oral composition in an amount from 180 mg/10 mL to 250 mg/10 mL. In some embodiments, the at least one antioxidant is present in the oral composition in an amount from 190 mg/10 mL to 250 mg/10 mL. In some embodiments, the at least one antioxidant is present in the oral composition in an amount from 200 mg/10 mL to 250 mg/10 mL. In some embodiments, the at least one antioxidant is present in the oral composition in an amount from 210 mg/10 mL to 250 mg/10 mL. In some embodiments, the at least one antioxidant is present in the oral composition in an amount from 220 mg/10 mL to 250 mg/10 mL. In some embodiments, the at least one antioxidant is present in the oral composition in an amount from 230 mg/10 mL to 250 mg/10 mL. In some embodiments, the at least one antioxidant is present in the oral composition in an amount from 240 mg/10 mL to 250 mg/10 mL. In some embodiments, the at least one antioxidant is present in the oral composition in an amount from 0.1 mg/10 mL to 30 mg/10 mL.

In some embodiments, the oral composition comprises from 1 mg/10 mL to 20 mg/10 mL ascorbyl palmitate. In some embodiments, the oral composition comprises 8 mg/10 mL ascorbyl palmitate.

In some embodiments, the oral composition comprises from 5 mg/10 mL to 30 mg/10 mL alpha-tocopherol. In some embodiments, the oral composition comprises 20 mg/10 mL alpha-tocopherol.

In some embodiments, the oral composition comprises 5 mg/10 mL to 30 mg/10 mL lecithin.

In some embodiments, the oral composition comprises from 1 mg/10 mL to 250 mg/10 mL ascorbyl palmitate, from 1 mg/10 mL to 50 mg/10 mL alpha-tocopherol, and from 1 mg/10 mL to 50 mg/10 mL lecithin. In some embodiments, the oral composition comprises from 150 mg/10 mL to 200 mg/10 mL ascorbyl palmitate, from 5 mg/10 mL to 30 mg/10 mL alpha-tocopherol, and from 5 mg/10 mL to 30 mg/10 mL lecithin.

### Preservatives

In some embodiments, the oral composition of the present disclosure (e.g., oral pharmaceutical composition or dietary supplement) may comprise at least one preservative.

In some embodiments, the oral composition comprises a preservative selected from a quaternary ammonium compound (such as benzalkonium chloride, benzethonium chloride, cetrimide, cetylpyridinium chloride, lauralkonium chloride and myristyl picolinium chloride), a mercurial agent (such as phenylmercuric nitrate, phenylmercuric acetate and thimerosal), an alcoholic agent (such as chlorobutanol, phenylethyl alcohol and benzyl alcohol), an antibacterial ester (such as esters of p-hydroxybenzoic acid), a chelating agent (such as disodium edetate (EDTA)), chlorhexidine, chlorocresol, sorbic acid, a benzoate (such as sodium benzoate), a propionate (such as sodium proprionate), methylparaben, ethylparaben, propylparaben, butylparaben paraben, sulfur dioxide, sulfite, nitrite, nitrate, lactic acid, propionic acid, propionate, ascorbic acid, ascorbate, butylated hydroxytoluene (BHT), hydroxyanisole, propyl gallate, lavender, thyme, peppermint, cajuput, cinnamon, clove, eucalyptus, sage, tea tree, salts of the foregoing, and combinations thereof.

In some embodiments, the oral composition comprises methylparaben or propylparaben. In some embodiments, the composition comprises methylparaben and propylparaben. In some embodiments, the oral composition comprises a quaternary ammonium compound (such as benzalkonium chloride, benzethonium chloride, cetrimide, cetylpyridinium chloride, lauralkonium chloride and myristyl picolinium chloride). In some embodiments, the oral composition comprises a mercurial agent (such as phenylmercuric nitrate, phenylmercuric acetate and thimerosal). In some embodiments, the oral composition comprises an alcoholic agent (such as chlorobutanol, phenylethyl alcohol and benzyl alcohol). In some embodiments, the oral composition comprises an antibacterial ester (such as esters of p-hydroxybenzoic acid). In some embodiments, the oral composition comprises a chelating agent (such as disodium edetate (EDTA)). In some embodiments, the oral composition comprises chlorhexidine. In some embodiments, the oral composition comprises chlorocresol. In some embodiments, the oral composition comprises sorbic acid. In some embodiments, the oral composition comprises a benzoate (such as sodium benzoate). In some embodiments, the oral composition comprises a propionate (such as sodium proprionate). In some embodiments, the oral composition comprises ethylparaben. In some embodiments, the oral composition comprises butylparaben paraben. In some embodiments, the oral composition comprises sulfur dioxide. In some embodiments, the oral composition comprises sulfite. In some embodiments, the oral composition comprises nitrite. In some embodiments, the oral composition comprises nitrate. In some embodiments, the oral composition comprises lactic acid. In some embodiments, the oral composition comprises propionic acid. In some embodiments, the oral composition comprises propionate. In some embodiments, the oral composition comprises ascorbic acid. In some embodiments, the oral composition comprises ascorbate. In some embodiments, the oral composition comprises butylated hydroxytoluene (BHT). In some embodiments, the oral composition comprises hydroxyanisole. In some embodiments, the oral composition comprises propyl gallate. In some embodiments, the oral composition comprises lavender. In some embodiments, the oral composition comprises thyme. In some embodiments, the oral composition comprises peppermint. In some embodiments, the oral composition comprises cajuput. In some embodiments, the oral composition comprises cinnamon. In some embodiments, the oral composition comprises clove. In some embodiments, the oral composition comprises eucalyptus. In some embodiments, the oral composition comprises sage. In some embodiments, the oral composition comprises tea tree oil.

In some embodiments, the oral composition comprises a preservative selected from lavender, thyme, peppermint, cajuput, cinnamon, clove, eucalyptus, sage, or tea tree.

In some embodiments, the at least one preservative is present in the oral composition in an amount from 0.1 mg/10 mL to 50 mg/10 mL, such as from 0.1 mg/10 mL to 25 mg/10 mL or from 1 mg/10 mL to 10 mg/10 mL. In some embodiments, the at least one preservative is present in an amount from 0.1 mg/10 mL to 50 mg/10 mL. In some embodiments, the at least one preservative is present in an amount from about 0.1 mg/10 mL to 25 mg/10 mL. In some embodiments, the at least one preservative is present in an amount from about 0.1 mg/10 mL to 10 mg/10 mL.

In some embodiments, the oral composition does not contain a preservative. In some embodiments, the oral pharmaceutical composition does not contain a preservative. In some embodiments, the dietary supplement does not contain a preservative.

### Flavor Ingredients

In some embodiments, the oral composition of the present disclosure (e.g., oral pharmaceutical composition or dietary supplement) may contain at least one flavor ingredient.

In some embodiments, the oral composition comprises at least one flavor ingredient selected from citrus (such as orange, lemon, and grapefruit), mint, a masking agent (e.g., Optify™ Masking Flavor), vanillin, cinnamon, coconut, ginger, almond, menthol, grape, raspberry, cherry, pineapple, and combinations thereof.

In some embodiments, the oral composition comprises at least one flavor ingredient comprising lemon and mint. In some embodiments, the oral composition comprises at least one flavor ingredient comprising lemon, mint, and a masking agent (e.g., Optify™ Masking Flavor).

In some embodiments, the flavor ingredient is citrus. In some embodiments, the flavor ingredient is orange. In some embodiments, the flavor ingredient is lemon. In some embodiments, the flavor ingredient is grapefruit. In some embodiments, the flavor ingredient is lime. In some embodiments, the flavor ingredient is mint. In some embodiments, the flavor ingredient is a masking agent (e.g., Optify™ Masking Flavor). In some embodiments, the flavor ingredient is vanillin. In some embodiments, the flavor ingredient is cinnamon. In some embodiments, the flavor ingredient is coconut. In some embodiments, the flavor ingredient is ginger. In some embodiments, the flavor ingredient is almond. In some embodiments, the flavor ingredient is menthol. In some embodiments, the flavor ingredient is grape. In some embodiments, the flavor ingredient is raspberry. In some embodiments, the flavor ingredient is cherry. In some embodiments, the flavor ingredient is pineapple.

In some embodiments, the at least one flavor ingredient is present in the oral composition in an amount from 0.1 mg/10 mL to 1,000 mg/10 mL, such as from 0.1 mg/10 mL to 500 mg/10 mL, from 0.1 mg/10 mL to 300 mg/10 mL or from 100 mg/10 mL to 300 mg/10 mL. In some embodiments, the at least one flavor ingredient is present in the oral composition in an amount from 1 mg/10 mL to 50 mg/10 mL. In some embodiments, the at least one flavor ingredient is present in the oral composition in an amount from 5 mg/10 mL to 50 mg/10 mL. In some embodiments, the at least one flavor ingredient is present in the oral composition in an amount from 10 mg/10 mL to 50 mg/10 mL. In some embodiments, the at least one flavor ingredient is present in the oral composition in an amount from 20 mg/10 mL to 50 mg/10 mL. In some embodiments, the at least one flavor ingredient is present in the oral composition in an amount from 25 mg/10 mL to 50 mg/10 mL. In some embodiments, the at least one flavor ingredient is present in the oral composition in an amount of 27.2 mg/10 mL.

### Sweetener

In some embodiments, the oral composition of the present disclosure (e.g., oral pharmaceutical composition or dietary supplement) may contain at least one sweetener.

In some embodiments, the sweetener is selected from stevia and isolated compounds therefrom (such as rebaudiosides A, D, M, B, AM and N), monk fruit and isolated compounds therefrom (mogroside V, siamenoside, mogroside IV, mogroside Ille), protein sweeteners and variants thereof (thaumatin, brazzein, Amai protein, sweet truffle protein), sucralose, potassium acesulfame, aspartame, neotame, advantame, cyclamate, saccharin, and combinations thereof.

In some embodiments, the sweetener is or comprises saccharin.

In some embodiments, the sweetener is rebaudioside A. In some embodiments, the sweetener is rebaudioside B. In some embodiments, the sweetener is rebaudioside D. In some embodiments, the sweetener is rebaudioside M. In some embodiments, the sweetener is rebaudioside AM. In some embodiments, the sweetener is rebaudioside N. In some embodiments, the sweetener derived from monk fruit. In some embodiments, the sweetener is mogroside V. In some embodiments, the sweetener is mogroside IV. In some embodiments, the sweetener is mogroside Ille. In some embodiments, the sweetener is siamenoside. In some embodiments, the sweetener is thaumatin. In some embodiments, the sweetener is brazzein. In some embodiments, the sweetener is amai protein. In some embodiments, the sweetener is sweet truffle protein. In some embodiments, the sweetener is sucralose. In some embodiments, the sweetener is potassium acesulfame. In some embodiments, the sweetener is aspartame. In some embodiments, the sweetener is neotame. In some embodiments, the sweetener is advantame. In some embodiments, the sweetener is cyclamate. In some embodiments, the sweetener is saccharin.

In some embodiments, the at least one sweetener is present in the oral composition in an amount from 0.1 mg/10 mL to 50 mg/10 mL, such as from 0.1 mg/10 mL to 25 mg/10 mL or from 1 mg/10 mL to 10 mg/10 mL. In some embodiments, the at least one sweetener is present in the oral composition in an amount from 0.1 mg/10 mL to 25 mg/10 mL. In some embodiments, the at least one sweetener is present in the oral composition in an amount from 1 mg/10 mL to 10 mg/10 mL.

### EMBODIMENTS

In some embodiments, an oral composition of the present disclosure comprises, per 10 mL, from 3,600 mg to 4,000 mg total omega-3 fatty acid ethyl esters, wherein the sum of EPA n-3 EE and DHA n-3 EE represent from 3,000 mg to 4,000 mg. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof, at least one flavor ingredient selected from lemon, mint, a masking agent (e.g., Optify™ Masking Flavor), and a combination thereof, at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 g/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is an oral pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters, wherein the sum of EPA n-3 EE and DHA n-3 EE represent from 2,000 mg to 4,500 mg. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof, at least one flavor ingredient selected from lemon, mint, a masking agent (e.g., Optify™ Masking Flavor), and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters, wherein the sum of EPA n-3 EE and DHA n-3 EE represent from 2,000 mg to 4,500 mg, and wherein the oral composition does not comprise a preservative. The oral composition can further comprise at least one antioxidant, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one flavor ingredient selected from lemon, mint, a masking agent (e.g., Optify™ Masking Flavor), and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is an oral pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters, wherein the weight ratio of EPA n-3 EE to DHA n-3 EE is from 2:1 to 1:1. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof, at least one preservative selected from methylparaben, propylparaben, and a combination thereof; at least one flavor ingredient selected from lemon, mint, a masking agent (e.g., Optify™ Masking Flavor), and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters, wherein the weight ratio of EPA n-3 EE to DHA n-3 EE is from 2:1 to 1:1, and wherein the pharmaceutical composition does not comprise a preservative. The oral composition can further comprise at least one antioxidant, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one flavor ingredient selected from lemon, mint, a masking agent (e.g., Optify™ Masking Flavor), and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof, from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters, wherein the sum of EPA n-3 EE and DHA n-3 EE represent from 2,000 mg to 4,500 mg and the weight ratio of EPA n-3 EE to DHA n-3 EE is from 2:1 to 1:1. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof; at least one flavor ingredient selected from lemon, mint, a masking agent (e.g., Optify™ Masking Flavor), and a combination thereof, at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters, wherein the sum of EPA n-3 EE and DHA n-3 EE represent from 2,000 mg to 4,500 mg, the weight ratio of EPA n-3 EE to DHA n-3 EE is from 2:1 to 1:1, and oral composition does not comprise a preservative. The oral composition can further comprise at least one antioxidant, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof, at least one flavor ingredient selected from lemon, mint, a masking agent (e.g., Optify™ Masking Flavor), and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters and less than 6% omega-6 fatty ethyl esters relative to the total omega-3 fatty acid ethyl esters. In some embodiments, an oral composition of the present disclosure comprises, per 10 mL, from 3,000 mg to 5,000 mg total omega-3 fatty acid ethyl esters and less than 5% omega-6 fatty ethyl esters relative to the total omega-3 fatty acid ethyl esters. In some embodiments, an oral composition of the present disclosure comprises, per 10 mL, from 3,000 mg to 5,000 mg total omega-3 fatty acid ethyl esters and less than 4% omega-6 fatty ethyl esters relative to the total omega-3 fatty acid ethyl esters. In some embodiments, an oral composition of the present disclosure comprises, per 10 mL, from 3,000 mg to 5,000 mg total omega-3 fatty acid ethyl esters and less than 3% omega-6 fatty ethyl esters relative to the total omega-3 fatty acid ethyl esters. In some embodiments, an oral composition of the present disclosure comprises, per 10 mL, from 3,000 mg to 5,000 mg total omega-3 fatty acid ethyl esters and less than 2% omega-6 fatty ethyl esters relative to the total omega-3 fatty acid ethyl esters. In some embodiments, an oral composition of the present disclosure comprises, per 10 mL, from 3,000 mg to 5,000 mg omega-3 total fatty acid ethyl esters and less than 1.5% omega-6 fatty ethyl esters relative to the total omega-3 fatty acid ethyl esters. In some embodiments, an oral composition of the present disclosure comprises, per 10 mL, from 3,000 mg to 5,000 mg total omega-3 fatty acid ethyl esters and less than 1% omega-6 fatty ethyl esters relative to the total omega-3 fatty acid ethyl esters. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof, at least one flavor ingredient selected from lemon, mint, a masking agent (e.g., Optify™ Masking Flavor), and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof, from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters and less than 6% omega-6 fatty ethyl esters relative to the total omega-3 fatty acid ethyl esters, wherein the sum of EPA n-3 EE and DHA n-3 EE represent from 2,000 mg to 4,500 mg. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof, at least one flavor ingredient selected from lemon, mint, a masking agent (e.g., Optify™ Masking Flavor), and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters and less than 6% omega-6 fatty ethyl esters relative to the total omega-3 fatty acid ethyl esters, wherein the sum of EPA n-3 EE and DHA n-3 EE represent from 2,000 mg to 4,500 mg, and wherein the oral composition does not comprise a preservative. The oral composition can further comprise at least one antioxidant, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one flavor ingredient selected from lemon, mint, a masking agent (e.g., Optify™ Masking Flavor), and a combination thereof, at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters and less than 6% omega-6 fatty ethyl esters relative to the total omega-3 fatty acid ethyl esters, wherein the weight ratio of EPA n-3 EE to DHA n-3 EE is from 2:1 to 1:1. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof; at least one flavor ingredient selected from lemon, mint, a masking agent (e.g., Optify™ Masking Flavor), and a combination thereof; at least one sweetener comprising saccharin; and a liquid carrier comprising olive oil (e.g., refined grade IV olive oil). In more particular embodiments, the oral pharmaceutical comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters and less than 6% omega-6 fatty ethyl esters relative to the total omega-3 fatty acid ethyl esters, wherein the sum of EPA n-3 EE and DHA n-3 EE represent from 2,000 mg to 4,500 mg and the weight ratio of EPA n-3 EE to DHA n-3 EE is from 2:1 to 1:1. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof, at least one flavor ingredient selected from lemon, mint, a masking agent (e.g., Optify™ Masking Flavor), and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises a fatty acid composition that comprises at least 90% by weight total omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE), wherein the oral composition comprises from 3,000 mg to 5,000 mg total omega-3 fatty acid ethyl esters per 10 mL. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof, at least one flavor ingredient selected from lemon, mint, a masking agent (e.g., Optify™ Masking Flavor), and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises a fatty acid composition that comprises at least 90% by weight total omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE), wherein the oral composition comprises from 3,000 mg to 5,000 mg total omega-3 fatty acid ethyl esters per 10 mL, and wherein the oral composition does not comprise a preservative. The oral composition can further comprise at least one antioxidant, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one flavor ingredient selected from lemon, mint, a masking agent (e.g., Optify™ Masking Flavor), and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises a fatty acid composition that comprises at least 90% by weight total omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE), wherein the oral composition comprises from 3,000 mg to 5,000 mg total omega-3 fatty acid ethyl esters per 10 mL, and wherein the sum of EPA n-3 EE and DHA n-3 EE represent from 2,000 mg to 4,500 mg. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof; at least one flavor ingredient selected from lemon, mint, a masking agent (e.g., Optify™ Masking Flavor), and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises a fatty acid composition that comprises at least 90% by weight total omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE), wherein the oral composition comprises from 3,000 mg to 5,000 mg total omega-3 fatty acid ethyl esters per 10 mL, and wherein the weight ratio of EPA n-3 EE to DHA n-3 EE is from 2:1 to 1:1. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof, at least one flavor ingredient selected from lemon, mint, a masking agent (e.g., Optify™ Masking Flavor), and a combination thereof, at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises a fatty acid composition that comprises at least 90% by weight total omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE), wherein the oral composition comprises from 3,000 mg to 5,000 mg total omega-3 fatty acid ethyl esters per 10 mL, wherein the sum of EPA n-3 EE and DHA n-3 EE represent from 2,000 mg to 4,500 mg, and wherein the weight ratio of EPA n-3 EE to DHA n-3 EE is from 2:1 to 1:1. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof; at least one flavor ingredient selected from lemon, mint, and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises a fatty acid composition that comprises at least 90% by weight total omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE), wherein the oral composition comprises from 3,000 mg to 5,000 mg total omega-3 fatty acid ethyl esters per 10 mL, wherein the sum of EPA n-3 EE and DHA n-3 EE represent from 2,000 mg to 4,500 mg, wherein the weight ratio of EPA n-3 EE to DHA n-3 EE is from 2:1 to 1:1, and wherein the oral composition comprises less than 6% omega-6 fatty ethyl esters relative to the total omega-3 fatty acid ethyl esters. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof; at least one flavor ingredient selected from lemon, mint, and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof, from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters, wherein the sum of EPA n-3 EE and DHA n-3 EE represent from 2,000 mg to 4,500 mg. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof, at least one flavor ingredient selected from lemon, mint, and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof, from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg omega-3 total fatty acid ethyl esters, wherein the weight ratio of EPA n-3 EE to DHA n-3 EE is from 2:1 to 1:1. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof; at least one flavor ingredient selected from lemon, mint, and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters, wherein the sum of EPA n-3 EE and DHA n-3 EE represent from 2,000 mg to 4,500 mg and the weight ratio of EPA EE to DHA EE is from 2:1 to 1:1. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof, at least one flavor ingredient selected from lemon, mint, and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters and less than 6% omega-6 fatty ethyl esters relative to the total omega-3 fatty acid ethyl esters. In some embodiments, an oral composition of the present disclosure comprises, per 10 mL, from 3,000 mg to 5,000 mg total omega-3 fatty acid ethyl esters and less than 5% omega-6 fatty ethyl esters relative to the total omega-3 fatty acid ethyl esters. In some embodiments, an oral composition of the present disclosure comprises, per 10 mL, from 3,000 mg to 5,000 mg total omega-3 fatty acid ethyl esters and less than 4% omega-6 fatty ethyl esters relative to the total omega-3 fatty acid ethyl esters. In some embodiments, an oral composition of the present disclosure comprises, per 10 mL, from 3,000 mg to 5,000 mg total omega-3 fatty acid ethyl esters and less than 3% omega-6 fatty ethyl esters relative to the total omega-3 fatty acid ethyl esters. In some embodiments, an oral composition of the present disclosure comprises, per 10 mL, from 3,000 mg to 5,000 mg total omega-3 fatty acid ethyl esters and less than 2% omega-6 fatty ethyl esters relative to the total omega-3 fatty acid ethyl esters. In some embodiments, an oral composition of the present disclosure comprises, per 10 mL, from 3,000 mg to 5,000 mg total omega-3 fatty acid ethyl esters and less than 1.5% omega-6 fatty ethyl esters relative to the total omega-3 fatty acid ethyl esters. In some embodiments, an oral composition of the present disclosure comprises, per 10 mL, from 3,000 mg to 5,000 mg omega-3 total fatty acid ethyl esters and less than 1% omega-6 fatty ethyl esters relative to the total omega-3 fatty acid ethyl esters. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof; at least one flavor ingredient selected from lemon, mint, and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters and less than 6% omega-6 fatty ethyl esters relative to the total omega-3 fatty acid ethyl esters, wherein the sum of EPA n-3 EE and DHA n-3 EE represent from 2,000 mg to 4,500 mg. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof, at least one preservative selected from methylparaben, propylparaben, and a combination thereof; at least one flavor ingredient selected from lemon, mint, and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters and less than 6% omega-6 fatty ethyl esters relative to the total omega-3 fatty acid ethyl esters, wherein the weight ratio of EPA n-3 EE to DHA n-3 EE is from 2:1 to 1:1. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof; at least one flavor ingredient selected from lemon, mint, and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises, per 10 mL, from 3,000 mg to 5,000 mg total omega-3 fatty acid ethyl esters and less than 6% omega-6 fatty ethyl esters relative to the total omega-3 fatty acid ethyl esters, wherein the sum of EPA n-3 EE and DHA n-3 EE represent from 2,000 mg to 4,500 mg and the weight ratio of EPA n-3 EE to DHA n-3 EE is from 2:1 to 1:1. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof, at least one flavor ingredient selected from lemon, mint, and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises a fatty acid composition that comprises at least 90% by weight for the total of omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE), and from 3,000 mg to 5,000 mg total omega-3 fatty acid ethyl esters per 10 mL. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof, at least one flavor ingredient selected from lemon, mint and a combination thereof, at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises a fatty acid composition that comprises at least 90% by weight for the total of omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE), and from 3,000 mg to 5,000 mg total omega-3 fatty acid ethyl esters per 10 mL, and wherein the oral composition does not comprise a preservative. The oral composition can further comprise at least one antioxidant, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one flavor ingredient selected from lemon, mint, a masking agent (e.g., Optify™ Masking Flavor), and a combination thereof, at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 1,000 mg/10 mL at least one flavor ingredient, wherein the at least one flavor ingredient is selected from lemon, mint, a masking agent (e.g., Optify™ Masking Flavor), and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises a fatty acid composition that comprises at least 90% by weight for the total of omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE), wherein the oral composition comprises from 3,000 mg to 5,000 mg total omega-3 fatty acid ethyl esters per 10 mL, and wherein the sum of EPA n-3 EE and DHA n-3 EE represent from 2,000 mg to 4,500 mg. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof, at least one flavor ingredient selected from lemon, mint, and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises a fatty acid composition that comprises at least 90% by weight for the total of omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE), wherein the oral composition comprises from 3,000 mg to 5,000 mg total omega-3 fatty acid ethyl esters per 10 mL, and wherein the weight ratio of EPA n-3 EE to DHA n-3 EE is from 2:1 to 1:1. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof; at least one flavor ingredient selected from lemon, mint, and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises a fatty acid composition that comprises at least 90% by weight for the total of omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE), wherein the oral composition comprises from 3,000 mg to 5,000 mg total omega-3 fatty acid ethyl esters per 10 mL, wherein the sum of EPA n-3 EE and DHA n-3 EE represent from 2,000 mg to 4,500 mg, and wherein the weight ratio of EPA n-3 EE to DHA n-3 EE is from 2:1 to 1:1. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof; at least one preservative selected from methylparaben, propylparaben, and a combination thereof; at least one flavor ingredient selected from lemon, mint, and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

In some embodiments, an oral composition of the present disclosure comprises a fatty acid composition that comprises at least 90% by weight for the total of omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE), wherein the oral composition comprises from 3,000 mg to 5,000 mg total omega-3 fatty acid ethyl esters per 10 mL, wherein the sum of EPA n-3 EE and DHA n-3 EE represent from 2,000 mg to 4,500 mg, wherein the weight ratio of EPA n-3 EE to DHA n-3 EE is from 2:1 to 1:1, and wherein the oral composition comprises less than 6% omega-6 fatty ethyl esters relative to the total omega-3 fatty acid ethyl esters. The oral composition can further comprise at least one antioxidant, at least one preservative, at least one flavor ingredient, a liquid carrier, or a combination thereof. In more particular embodiments, the oral composition further comprises at least one antioxidant selected from ascorbyl palmitate, lecithin, alpha-tocopherol, and combinations thereof, at least one preservative selected from methylparaben, propylparaben, and a combination thereof; at least one flavor ingredient selected from lemon, mint, and a combination thereof; at least one sweetener comprising saccharin; and a liquid carrier comprising olive oil (e.g., refined grade IV olive oil). In more particular embodiments, the oral composition comprises from 0.1 mg/10 mL to 250 mg/10 mL at least one antioxidant, from 0.1 mg/10 mL to 50 mg/10 mL at least one preservative, from 0.1 mg/10 mL to 1,000 mg/10 mL at least one flavor ingredient, and/or from 0.1 mg/10 mL to 50 mg/10 mL at least one sweetener. In more particular embodiments, the oral composition comprises from 0.1 mg/10 mL to 250 mg/10 mL at least one antioxidant, wherein the at least one antioxidant is selected from ascorbyl palmitate, lecithin, alpha-tocopherol, and combinations thereof; from 0.1 mg/10 mL to 50 mg/10 mL at least one preservative, wherein the at least one preservative is selected from methylparaben, propylparaben, and a combination thereof; from 0.1 mg/10 mL to 1,000 mg/10 mL at least one flavor ingredient, wherein the at least one flavor ingredient is selected from lemon, mint, and a combination thereof; from 0.1 mg/10 mL to 50 mg/10 mL at least one sweetener, wherein the sweetener is saccharin, and a liquid carrier, wherein the liquid carrier comprises or is refined (grade IV) olive oil. In some embodiments, the oral composition is a pharmaceutical composition. In some embodiments, the oral composition is a dietary supplement.

### Unit Dose

In one aspect, the present disclosure also provides a unit dose of the oral composition described herein of a distinct volume. In some embodiments, a unit dose comprises from 1 mL to 100 mL of the oral composition of the present disclosure, such as from 1 mL to 90 mL, from 1 mL to 80 mL, from 1 mL to 70 mL, from 1 mL to 60 mL, from 1 mL to 50 mL, from 1 mL to 40 mL, from 1 mL to 30 mL, from 1 mL to 20 mL, from 1 mL to 10 mL, or from 1 mL to 5 mL. In some embodiments, a unit dose comprises from 1 mL to 20 mL of the oral composition of the present disclosure, such as from 1 mL to 15 mL, from 1 mL to 10 mL, from 1 mL to 5 mL, from 5 mL to 20 mL, from 5 mL to 15 mL, from 5 mL to 10 mL, from 10 mL to 20 mL, from 10 mL to 15 mL, or from 15 mL to 20 mL. In some embodiments, a unit dose comprises from 2 mL to 7 mL of the oral composition of the present disclosure, such as 2 mL, 3 mL, 4 mL, 5 mL, 6 mL or 7 mL. In some embodiments, a unit dose comprises from 8 mL to 12 mL of the oral composition of the present disclosure, such as 8 mL, 9 mL, 10 mL, 11 mL, or 12 mL. In some embodiments, a unit dose comprises 5 mL of the oral composition of the present disclosure. In some embodiments, a unit dose comprises 10 mL of the oral composition of the present disclosure.

In some embodiments, a 10 mL unit dose of an oral composition of the present disclosure comprises from 3,000 mg to 5,000 mg total omega-3 fatty acid ethyl esters and a liquid carrier, wherein the liquid carrier comprises or is refined (grade IV) olive oil. In more particular embodiments, the 10 mL unit dose of the oral composition has one or more of: a sum of EPA n-3 EE and DHA n-3 EE from 2,000 mg to 4,500 mg; a weight ratio of EPA n-3 EE to DHA n-3 EE from 2:1 to 1:1; less than 6% omega-6 fatty ethyl esters relative to the total omega-3 fatty acid ethyl esters; and at least 90% by weight total omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE). The 10 mL unit dose of the oral composition can further comprise at least one antioxidant, at least one preservative, at least one flavor ingredient, or a combination thereof. In more particular embodiments, the 10 mL unit dose of the oral composition further comprises at least one antioxidant selected from ascorbyl palmitate, lecithin, alpha-tocopherol, and combinations thereof; at least one preservative selected from methylparaben, propylparaben, and a combination thereof, at least one flavor ingredient selected from lemon, mint, a masking agent (e.g., Optify™ Masking Flavor), and a combination thereof; and at least one sweetener comprising saccharin. In more particular embodiments, the 10 mL unit dose of the oral composition further comprises from 0.1 mg to 250 mg at least one antioxidant, from 0.1 mg to 50 mg at least one preservative, from 0.1 mg to 1,000 mg at least one flavor ingredient, and from 0.1 mg to 50 mg at least one sweetener. In more particular embodiments, the 10 mL unit dose of the oral composition further comprises from 0.1 mg to 250 mg at least one antioxidant, wherein the at least one antioxidant is selected from ascorbyl palmitate, lecithin, alpha-tocopherol, and combinations thereof; from 0.1 mg to 50 mg at least one preservative, wherein the at least one preservative is selected from methylparaben, propylparaben, and a combination thereof; from 0.1 mg to 1,000 mg at least one flavor ingredient, wherein the at least one flavor ingredient is selected from lemon, mint, a masking agent (e.g., Optify™ Masking Flavor), and a combination thereof; and from 0.1 mg to 50 mg at least one sweetener, wherein the sweetener is saccharin. In some embodiments, the 10 mL unit dose comprises an oral pharmaceutical composition of the present disclosure.

In some embodiments, a 10 mL unit dose of an oral composition of the present disclosure comprises from 3,000 mg to 5,000 mg total omega-3 fatty acid ethyl esters and a liquid carrier, wherein the liquid carrier comprises or is refined (grade IV) olive oil, and wherein the oral composition does not comprise a preservative. In more particular embodiments, the 10 mL unit dose of the oral composition has one or more of: a sum of EPA n-3 EE and DHA n-3 EE from 2,000 mg to 4,500 mg; a weight ratio of EPA n-3 EE to DHA n-3 EE from 2:1 to 1:1; less than 6% omega-6 fatty ethyl esters relative to the total omega-3 fatty acid ethyl esters; and at least 90% by weight total omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE). The 10 mL unit dose of the oral composition can further comprise at least one antioxidant, at least one flavor ingredient, or a combination thereof. In more particular embodiments, the 10 mL unit dose of the oral composition further comprises at least one antioxidant selected from ascorbyl palmitate, lecithin, alpha-tocopherol, and combinations thereof; at least one flavor ingredient selected from lemon, mint, a masking agent (e.g., Optify™ Masking Flavor), and a combination thereof; and at least one sweetener comprising saccharin. In more particular embodiments, the 10 mL unit dose of the oral composition further comprises from 0.1 mg to 250 mg at least one antioxidant, from 0.1 mg to 1,000 mg at least one flavor ingredient, and from 0.1 mg to 50 mg at least one sweetener. In more particular embodiments, the 10 mL unit dose of the oral composition of the present disclosure further comprises from 0.1 mg to 250 mg at least one antioxidant, wherein the at least one antioxidant is selected from ascorbyl palmitate, lecithin, alpha-tocopherol, and combinations thereof; from 0.1 mg to 1,000 mg at least one flavor ingredient, wherein the at least one flavor ingredient is selected from lemon, mint, a masking agent (e.g., Optify™ Masking Flavor), and a combination thereof; and from 0.1 mg to 50 mg at least one sweetener, wherein the sweetener is saccharin. In some embodiments, the 10 mL unit dose comprises an oral pharmaceutical composition of the present disclosure.

In some embodiments, a 5 mL unit dose of an oral composition of the present disclosure comprises from 1,500 mg to 2,500 mg total omega-3 fatty acid ethyl esters and a liquid carrier, wherein the liquid carrier comprises or is refined (grade IV) olive oil. In more particular embodiments, the 5 mL unit dose of the oral composition has one or more of: a sum of EPA n-3 EE and DHA n-3 EE from 1,000 mg to 2,250 mg; a weight ratio of EPA n-3 EE to DHA n-3 EE from 2:1 to 1:1; less than 6% omega-6 fatty ethyl esters relative to the total omega-3 fatty acid ethyl esters; and at least 90% by weight total omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE). The 5 mL unit dose of the oral composition can further comprise at least one antioxidant, at least one preservative, at least one flavor ingredient, or a combination thereof. In more particular embodiments, the 5 mL unit dose of the oral composition further comprises at least one antioxidant selected from ascorbyl palmitate, lecithin, alpha-tocopherol, and combinations thereof; at least one preservative selected from methylparaben, propylparaben, and a combination thereof; at least one flavor ingredient selected from lemon, mint, a masking agent (e.g., Optify™ Masking Flavor), and a combination thereof; and at least one sweetener comprising saccharin. In more particular embodiments, the 5 mL unit dose of the oral composition further comprises from 0.1 mg to 125 mg at least one antioxidant, from 0.1 mg to 25 mg at least one preservative, from 0.1 mg to 500 mg at least one flavor ingredient, and from 0.1 mg to 25 mg at least one sweetener. In more particular embodiments, the 5 mL unit dose of the oral composition further comprises from 0.1 mg to 125 mg at least one antioxidant, wherein the at least one antioxidant is selected from ascorbyl palmitate, lecithin, alpha-tocopherol, and combinations thereof; from 0.1 mg to 25 mg at least one preservative, wherein the at least one preservative is selected from methylparaben, propylparaben, and a combination thereof; from 0.1 mg to 500 mg at least one flavor ingredient, wherein the at least one flavor ingredient is selected from lemon, mint, a masking agent (e.g., Optify™ Masking Flavor), and a combination thereof; and from 0.1 mg to 25 mg at least one sweetener, wherein the sweetener is saccharin. In some embodiments, the 5 mL unit dose comprises a dietary supplement of the present disclosure.

In some embodiments, a 5 mL unit dose of an oral composition of the present disclosure comprises from 1,500 mg to 2,500 mg total omega-3 fatty acid ethyl esters and a liquid carrier, wherein the liquid carrier comprises or is refined (grade IV) olive oil, and wherein the oral composition does not comprise a preservative. In more particular embodiments, the 5 mL unit dose of the oral composition has one or more of: a sum of EPA n-3 EE and DHA n-3 EE from 1,000 mg to 2,250 mg; a weight ratio of EPA n-3 EE to DHA n-3 EE from 2:1 to 1:1; less than 6% omega-6 fatty ethyl esters relative to the total omega-3 fatty acid ethyl esters; and at least 90% by weight total omega-3 fatty acid ethyl esters (i.e., the sum of EPA n-3 EE, DHA n-3 EE, SDA n-3 EE, HPA n-3 EE, DPA n-3 EE, ETA n-3 EE, and ALA n-3 EE). The 5 mL unit dose of the oral composition can further comprise at least one antioxidant, at least one flavor ingredient, or a combination thereof. In more particular embodiments, the 5 mL unit dose of the oral composition further comprises at least one antioxidant selected from ascorbyl palmitate, lecithin, alpha-tocopherol, and combinations thereof; at least one flavor ingredient selected from lemon, mint, a masking agent (e.g., Optify™ Masking Flavor), and a combination thereof; and at least one sweetener comprising saccharin. In more particular embodiments, the 5 mL unit dose of the oral composition further comprises from 0.1 mg to 125 mg at least one antioxidant, from 0.1 mg to 500 mg at least one flavor ingredient, and from 0.1 mg to 25 mg at least one sweetener. In more particular embodiments, the 5 mL unit dose of the oral composition further comprises from 0.1 mg to 125 mg at least one antioxidant, wherein the at least one antioxidant is selected from ascorbyl palmitate, lecithin, alpha-tocopherol, and combinations thereof; from 0.1 mg to 500 mg at least one flavor ingredient, wherein the at least one flavor ingredient is selected from lemon, mint, a masking agent (e.g., Optify™ Masking Flavor), and a combination thereof; and from 0.1 mg to 25 mg at least one sweetener, wherein the sweetener is saccharin. In some embodiments, the 5 mL unit dose comprises a dietary supplement of the present disclosure.

### Packaging Materials

In some embodiments, the oral composition of the present disclosure (e.g., oral pharmaceutical composition or dietary supplement) is packaged in a polyethylene terephthalate (PET) bottle fitted with a closure, such as a dark amber PET bottle fitted with a closure. In some embodiments, the oral composition is packaged in a polypropylene (PP) bottle fitted with closure. In some embodiments, the oral composition is packaged in a high-density polyethylene (HDPE) bottle. In some embodiments, the bottle fitted with closure is translucent. In some embodiments, the bottle fitted with closure is opaque.

### Inert Headspace Gas

In some embodiments, the oral composition is packaged in a closed bottle with an inert gas (e.g., nitrogen gas, helium gas, or argon gas). In some embodiments, the oral composition is packaged in a closed bottle without the presence of an inert gas (normal atmosphere).

### Peroxide Values

Peroxidation of a fat or oil is a self-propagating chain reaction that occurs when the fat or oil is exposed to an oxidative load (e.g., oxygen gas and light). A peroxide value (PV) associated with a fat or oil is a measure of primary oxidation products. Unsaturated fatty acid esters are vulnerable to peroxidation. Variables that contribute to peroxidation include oxygen gas concentrations, temperature, and light exposure (e.g., sunlight and/or artificial light).

In some embodiments, the peroxide value of the oral composition of the present disclosure (e.g., oral pharmaceutical composition or dietary supplement) is less than 20 mEq/kg. In some embodiments, the peroxide value of the oral composition is less than 18 mEq/kg. In some embodiments, the peroxide value of the oral composition is less than 16 mEq/kg. In some embodiments, the peroxide value of the oral composition is less than 14 mEq/kg. In some embodiments, the peroxide value of the oral composition is less than 12 mEq/kg. In some embodiments, the peroxide value of the oral composition is less than 10 mEq/kg. In some embodiments, the peroxide value of the oral composition is less than 9 mEq/kg. In some embodiments, the peroxide value of the oral composition is less than 8 mEq/kg. In some embodiments, the peroxide value of the oral composition is less than 7 mEq/kg. In some embodiments, the peroxide value of the oral composition is less than 6 mEq/kg. In some embodiments, the peroxide value of the oral composition is less than 5 mEq/kg. In some embodiments, the peroxide value of the oral composition is less than 4 mEq/kg. In some embodiments, the peroxide value of the oral composition is less than 3 mEq/kg. In some embodiments, the peroxide value of the oral composition is less than 2 mEq/kg. In some embodiments, the peroxide value of the oral composition is less than 1 mEq/kg.

### Anisidine Values

An anisidine value (AV) is derived from a spectrophotometric measurement of secondary oxidation compounds (e.g., aldehydes) present in a fat or oil composition subject to some degree of oxidative load.

Anisidine value is a standardized test providing information about oxidative degradation of unsaturated fatty acids in fats, oil and other fatty acid containing products (e.g. methyl or ethyl esters of fatty acids). Double bonds in the unsaturated fatty acids undergo oxidation by oxygen that may result in complete cleavage of the double bond and formation of two aldehyde species. These aldehydes (products of oxidative degradation) are detected with the anisidine value test, that uses a condensation reaction of aldehydes with p-anisidine (p-methoxy aniline). The resulting Schiff bases are determined spectrophotometrically at 350 nm.

Positive response (interference) of excipients to the anisidine value test could be caused by presence of aldehydic compounds in the excipients. Some aldehydes could be present in olive oil as a result of oxidative degradation of olive oil unsaturated fatty acids, however these should not be considered as interferences and should be included into anisidine value of the product. Flavor chemical compositions often are not known, but some aldehydic compounds are known to be responsible for natural aroma of citrus species, e.g. citronellal, neral, perillaldehyde, C₈-C₁₂ aliphatic aldehydes, and it is likely that orange and lemon flavor placebo components can contain some aldehydic compounds. Since such aldehydes are not the result of unsaturated fatty acids oxidative degradation and are added intentionally (as a part of product flavor, taste and/or odor masking components), they represent interference to this test. In certain embodiments, the anisidine values provided below are for oral compositions without the flavor ingredient to eliminate such interference.

In some embodiments, the oral composition of the present disclosure (e.g., oral pharmaceutical composition or dietary supplement) has an anisidine value from 25 to 80, such as from 40 to 50, or from 40 to 45. In some embodiments, the oral composition has an anisidine value from 25 to 80. In some embodiments, the oral composition has an anisidine value from 40 to 50. In some embodiments, the oral composition has an anisidine value from 40 to 45. In some embodiments, the oral pharmaceutical composition has an anisidine value of not more than 85, such as not more than 75, not more than 65, not more than 55, not more than 45, not more than 35, not more than 25, or not more than 15.

### Stability

In some embodiments, the oral composition of the present disclosure (e.g., oral pharmaceutical composition or dietary supplement) exhibits improved stability over other omega-3 fatty acid ethyl ester containing compositions.

In some embodiments, the oral composition of the present disclosure (e.g., oral pharmaceutical composition or dietary supplement) exhibits a decrease in total omega-3 fatty acid ethyl ester content of 15% or less following storage at 25 °C±2 °C/60% RH±5% RH for 24 months, e.g. 12% or less or 10% or less. In some embodiments, the oral composition exhibits a decrease in total omega-3 fatty acid ethyl ester content of 15% or less following storage at 30 °C±2 °C/60% RH±5% RH for 24 months, e.g. 12% or less or 10% or less. In some embodiments, the oral composition exhibits a decrease in total omega-3 fatty acid ethyl ester content of 15% or less following storage at 40 C±2 °C/60% RH±5% RH for 24 months, e.g. 12% or less or 10% or less.

In some embodiments, the oral composition of the present disclosure (e.g., oral pharmaceutical composition or dietary supplement) exhibits a degradation of less than 2% with respect to EPA n-3 EE and DHA n-3 EE when subjected to conditions of the 10-day oxidation study.

In some embodiments, the oral composition of the present disclosure (e.g., oral pharmaceutical composition or dietary supplement) is a clear solution, i.e., the solution is free of visible particles. In some embodiments, the oral composition is observed as a clear solution (e.g., yellow) following storage at 25±C±2 °C/60% RH±5% RH for 24 months. In some embodiments, the oral composition is observed as a clear solution (e.g., yellow) following storage at 30 °C±2 °C/60% RH±5% RH for 24 months. In some embodiments, the oral composition is observed as a clear solution (e.g., yellow) following storage at 40 °C±2 °C/60% RH±5% RH for 24 months.

In some embodiments, the oral composition of the present disclosure (e.g., oral pharmaceutical composition or dietary supplement) exhibits not more than 5.0%/year percentage of weight loss following storage at 25 °C±2 °C/60% RH±5% RH for 24 months, such as less than 4.5%/year, less than 4.0%/year, less than 3.5%/year, less than 3.0%/year, less than 2.5%/year, less than 2.0%/year, less than 1.5%/year, less than 1.0%/year, or less than 0.5%/year. In some embodiments, the oral composition exhibits not more than 5.0%/year percentage of weight loss following storage at 30 °C±2 °C/60% RH±5% RH for 24 months, such as less than 4.5%/year, less than 4.0%/year, less than 3.5%/year, less than 3.0%/year, less than 2.5%/year, less than 2.0%/year, less than 1.5%/year, less than 1.0%/year, or less than 0.5%/year. In some embodiments, the oral composition exhibits not more than 5.0%/year percentage of weight loss following storage at 40 °C±2 °C/60% RH±5% RH for 24 months, such as less than 4.5%/year, less than 4.0%/year, less than 3.5%/year, less than 3.0%/year, less than 2.5%/year, less than 2.0%/year, less than 1.5%/year, less than 1.0%/year, or less than 0.5%/year.

In some embodiments, the oral composition of the present disclosure (e.g., oral pharmaceutical composition or dietary supplement) exhibits a color from 0.20 to 0.75 Absorbance Units at 400 nm following storage at 25 °C±2 °C/60% RH±5% RH for 24 months. In some embodiments, the oral composition exhibits a color from 0.20 to 0.75 Absorbance Units at 400 nm following storage at 30 °C±2 °C/60% RH±5% RH for 24 months. In some embodiments, the oral composition exhibits a color from 0.20 to 0.75 Absorbance Units at 400 nm following storage at 40 °C±2 °C/60% RH±5% RH for 24 months.

In some embodiments, the oral composition of the present disclosure (e.g., oral pharmaceutical composition or dietary supplement) has a specific gravity from 0.89 to 0.93 following storage at 25 °C±2 °C/60% RH±5% RH for 24 months. In some embodiments, the oral composition has a specific gravity from 0.89 to 0.93 following storage at 30 °C±2 °C/60% RH±5% RH for 24 months. In some embodiments, the oral composition has a specific gravity from 0.89 to 0.93 following storage at 40 °C±2 °C/60% RH±5% RH for 24 months.

In some embodiments, the oral composition of the present disclosure (e.g., oral pharmaceutical composition or dietary supplement) has an acid value of not more than 2.0 following storage at 25 °C±2 °C/60% RH±5% RH for 24 months, such as from 0.5 to 1.5. In some embodiments, the oral composition has an acid value of not more than 2.0 following storage at 30 °C±2 °C/60% RH±5% RH for 24 months, such as from 0.5 to 1.5. In some embodiments, the oral composition has an acid value of not more than 2.0 following storage at 40 °C±2 °C/60% RH±5% RH for 24 months, such as from 0.5 to 1.5.

In some embodiments, the oral composition of the present disclosure (e.g., oral pharmaceutical composition or dietary supplement) has an anisidine value of not more than 80 following storage at 25 °C±2 °C/60% RH±5% RH for 24 months, such as not more than 60, not more than 50, or from 35 to 60. In some embodiments, the oral composition has an anisidine value not more than 80 following storage at 30 °C±2 °C/60% RH±5% RH for 24 months, such as not more than 60, not more than 50, or from 35 to 60. In some embodiments, the oral composition has an anisidine value of not more than 80 following storage at 40 °C±2 °C/60% RH±5% RH for 24 months, such as not more than 60, not more than 50, or from 35 to 60.

In some embodiments, the oral composition of the present disclosure (e.g., oral pharmaceutical composition or dietary supplement) has a peroxide value of not more than 10 mEq/kg following storage at 25 °C±2 °C/60% RH±5% RH for 24 months, such as not more than 8 mEq/kg, not more than 5 mEq/kg, or from 2 to 4 mEq/kg. In some embodiments, the oral composition has a peroxide value of not more than 10 mEq/kg following storage at 30 °C±2 °C/60% RH±5% RH for 24 months, such as not more than 8 mEq/kg, not more than 5 mEq/kg, or from 2 to 4 mEq/kg. In some embodiments, the oral composition has a peroxide value of not more than 10 mEq/kg following storage at 40 °C±2 °C/60% RH±5% RH for 24 months, such as not more than 8 mEq/kg, not more than 5 mEq/kg, or from 2 to 4 mEq/kg.

### Methods of Use

In one aspect, the present disclosure provides a method of administering omega-3 fatty acid ethyl esters comprising orally administering an oral composition of the present disclosure (e.g., an oral pharmaceutical composition or a dietary supplement).

In another aspect, the present disclosure provides a method of reducing triglyceride levels in a subject in need thereof, the method comprising orally administering to the subject a therapeutically effective dose of an oral composition of the present disclosure. In some embodiments, the oral composition is an oral pharmaceutical composition.

In some embodiments, the subject has severe hypertriglyceridemia (HTG), i.e., triglyceride levels≥500 mg/dL. In some embodiments, the subject is on a lipid-lowering diet prior to a while taking the oral pharmaceutical composition of the present disclosure.

In another aspect, the present disclosure provides a method of providing cognitive support in a subject in need thereof, the method comprising orally administering to the subject a therapeutically effective dose of an oral composition of the present disclosure. In some embodiments, the oral composition is a dietary supplement. “Providing cognitive support” refers to a real or perceived improvement in mental clarity, focus, and memory.

In another aspect, the present disclosure provides a method of providing mood support in a subject in need thereof, the method comprising orally administering to the subject a therapeutically effective dose of an oral composition of the present disclosure. In some embodiments, the oral composition is a dietary supplement. “Providing mood support” refers to a real or perceived improvement mood balance and overall emotional well-being.

In another aspect, the present disclosure provides a method of improving heart health in a subject in need thereof, the method comprising orally administering to the subject a therapeutically effective dose of an oral composition of the present disclosure. In some embodiments, the oral composition is a dietary supplement. Improved heart health can be determined by a real or perceived improvement in cardiovascular function.

In another aspect, the present disclosure provides a method of improving joint health in a subject in need thereof, the method comprising orally administering to the subject a therapeutically effective dose of an oral composition of the present disclosure. In some embodiments, the oral composition is a dietary supplement. Improved joint health includes maintaining joint comfort or alleviating joint pain.

In another aspect, the present disclosure provides a method of improving eye health in a subject in need thereof, the method comprising orally administering to the subject a therapeutically effective dose of an oral composition of the present disclosure. In some embodiments, the oral composition is a dietary supplement. Improved eye health includes increased hydration to the eye.

In another aspect, the present disclosure provides a method of providing thermoregulation support in a subject in need thereof, the method comprising orally administering to the subject a therapeutically effective dose of an oral composition of the present disclosure. In some embodiments, the oral composition is a dietary supplement. Thermoregulation support can be determined by maintenance or improvement in temperature regulations, e.g., during mid-life transitions.

In another aspect, the present disclosure provides a method of providing hormonal balance support in a subject in need thereof, the method comprising orally administering to the subject a therapeutically effective dose of an oral composition of the present disclosure. In some embodiments, the oral composition is a dietary supplement.

In some embodiments, the subject is human. In some embodiments, the subject is a human female. In some embodiments, the subject is a peri-menopausal human female. In some embodiments, the subject is a menopausal human female.

In some embodiments, a dose of the oral composition of the present disclosure comprising from 3,000 mg to 5,000 mg omega-3 fatty acid ethyl esters per 10 mL is administered to the subject, such as from 3,200 to 4,800 mg omega-3 fatty acid ethyl esters per 10 mL, from 3,500 to 4,000 mg omega-3 fatty acid ethyl esters per 10 mL, or from 3,750 to 5,000 mg omega-3 fatty acid ethyl esters per 10 mL. In some embodiments, a dose of the oral composition of the present disclosure comprising from 3,800 mg to 4,200 mg omega-3 fatty acid ethyl esters per 10 mL, such as 4,000 mg omega-3 fatty acid ethyl esters is administered to the subject.

In some embodiments, a dose of the oral composition of the present disclosure has a volume from 8 mL to 15 mL, e.g., from 8 mL to 12 mL, 8 mL, 9 mL, 10 mL, 11 mL, 12 mL, 13 mL, 14 mL, or 15 mL. In some embodiments, a dose of the oral composition has a volume of 10 mL.

In some embodiments, a dose of the oral composition of the present disclosure having a volume from 8 mL to 15 mL and comprising from 3,000 mg to 5,000 mg omega-3 fatty acid ethyl esters is administered to the subject. In some embodiments, a dose of the oral composition of the present disclosure having a volume from 9 mL to 12 mL and comprising from 3,000 mg to 5,000 mg omega-3 fatty acid ethyl esters is administered to the subject. In some embodiments, a dose of the oral composition of the present disclosure having a volume of 10 mL and comprising from 3,000 mg to 5,000 mg omega-3 fatty acid ethyl esters is administered to the subject.

In some embodiments, a dose of the oral composition of the present disclosure has a volume from 3 mL to 7 mL, e.g., from 4 mL to 6 mL, 4 mL, 5 mL, 6 mL, or 6 mL. In some embodiments, a dose of the oral composition of the present disclosure has a volume of 5 mL.

In some embodiments, a dose of the oral composition of the present disclosure having a volume from 3 mL to 7 mL and comprising from 1,500 mg to 2,500 mg omega-3 fatty acid ethyl esters is administered to the subject. In some embodiments, a dose of the oral composition of the present disclosure having a volume from 4 mL to 6 mL and comprising from 1,500 mg to 2,500 mg omega-3 fatty acid ethyl esters is administered to the subject. In some embodiments, a dose of the oral composition of the present disclosure having a volume of 5 mL and comprising from 1,500 mg to 2,500 mg mg omega-3 fatty acid ethyl esters is administered to the subject.

In some embodiments, a dose of the oral composition of the present disclosure is administered orally to the subject at least once per day, e.g., 1 time, 2 times, 3 times, 4 times, or 5 or more times per day. In some embodiments, a dose of the oral composition of the present disclosure is administered orally to the subject once per day. In some embodiments, a dose of the oral composition of the present disclosure is administered orally to the subject twice per day.

In some embodiments, a dose of the oral composition of the present disclosure comprising from 3,000 mg to 5,000 mg omega-3 fatty acid ethyl esters is administered orally to the subject once per day. In some embodiments, a dose of the oral composition of the present disclosure comprising from 1,500 mg to 2,500 mg omega-3 fatty acid ethyl esters is administered orally to the subject once per day.

In some embodiments, a dose of the oral composition of the present disclosure is administered to a subject in two or more fractional doses over the course of a day (e.g., 5 mL twice a day; 2.5 mL four times a day; 1 mL ten times a day for a 10 mL dose).

### EXAMPLES

The following examples are for illustrative purposes only and should not be construed as limiting in any manner.

### Example 1 Production of Oral Pharmaceutical Composition (Solution)

To a stirring mixture of olive oil (grade IV, 33.166 kg) at 600 rpms at 80-100 °C was added saccharin (25 g) and the mixture was stirred at temperature for about 1 hour, until all solids had dissolved. With the temperature held at 80-100 °C, to the mixture was added ascorbyl palmitate (56 g) and stirred for another hour, until all solids were dissolved. The mixture was allowed to cool to 70 °C, followed by the addition of methylparaben (35 g) and its dissolution. This step took about 5 minutes. Care was taken to not exceed 70 °C Further to the mixture at 70 °C was added propylparaben (7 g), again taking care that the temperature not exceed 70 °C, and the mixture was stirred until dissolved, which took about 1 hour. While maintaining the temperature at 70 °C, lecithin was added and stirred until dissolved, approximately 1 hour. From here, the solution was allowed to cool to 30 °C, followed by the addition of alpha-tocopherol (142.8 g). The temperature was maintained at 30 °C with stirring until all dissolved, about 5 minutes. To this solution, lemon flavor (1.4 kg) was added, followed by lemon mint flavor (0.490 kg), and masking flavor (0.238 kg). To this stirring solution below 30 °C, omega-3 fatty acid ethyl esters (28 kg) was added. Once the addition was complete, the vessel was covered to exclude air and the headspace of the vessel was purged with nitrogen gas for 15 minutes.

### Analytical Methods

Omega-3-acid Ethyl Esters Method
Instrument: Agilent 8890 Gas Chromatograph (GC)
Software: Empower 3 Workstation
Column: HP Innowax Agilent 19091N-136IE (60 meters×250 mm×0.25 μm)
Carrier Gas: Helium
Split Flow: 20.0 mL/min
Septum Purge: 1.0 mL/min
Flow Rate: 1.0 mL/min
Split Injection Ratio: 20:1
Total Flow: 22.0 mL/min
Injector Temperature: 230 °C
Oven Temperature: Initial Temperature: 40 °C Hold Time: 5.0 minutes.
Gradient Temperature Ramp: 4 °C/minute.
Final Temperature: 230 °C Hold Time: 67.5 minutes.
Maximum Temperature: 250 °C
Total Run Time: 120.0 minutes
Injection Volume: 1 μL
Make-Up Gas Flow: 10 mL/min
Hydrogen Gas Flow: 30 mL/min
Air Flow: 300 mL/min
Diluent: Trimethylpentane (TMP)

### Example 2. Characterization of Omega-3 Ethyl Ester Oral Solution of the Present Disclosure

A highly purified omega-3 fatty acid ethyl ester mixture was obtained from KD Pharma Group.

### Omega-3 Fatty Acid Ethyl Ester Certificate of Analysis

| Parameter | Characteristics |
|---|---|
| Odor | Slight characteristic smell |
| Solubility | Practically insoluble in water. |
| | Very soluble in acetone, ethanol, heptane and methanol. |
| Density | Ca. 0.9 g/cm³ |

| Parameter | | Specification | Analysis | | Method |
|---|---|---|---|---|---|
| Identification EPA | | The retention times of the | The retention times of the | | In-house |
| ethyl esters and DHA | | peaks for eicosapentaenoic | peaks for eicosapentaenoic | | |
| ethyl esters | | acid ethyl ester in the Test | acid ethyl ester and | | |
| | | Solution correspond to | docosahexaenoic acid ethyl | | |
| | | those respective | ester in the Test Solution | | |
| | | compounds in the Standard | correspond to those | | |
| | | Solution, as obtained in the | respective compounds in | | |
| | | Assay. | | | |
| | | | the Standard Solution, as | | |
| | | | obtained in the Assay. | | |
| Appearance | | Pale Yellow Liquid | Pale Yellow Liquid | | Visual |
| Acid Value | | Max. 2.0 mg KOH/g | 0.3 | mg KOH/g | USP/Ph. Eur. |
| Peroxide Value | | Max. 10.0 meq O₂/kg | 1.6 | meq O₂/kg | USP/Ph. Eur. |
| Anisidine Value | | Max. 15 | ≤2.3 | | USP/Ph. Eur. |
| Absorbance | | Max. 0.55 AU at 233 nm | 0.34 | AU | USP/Ph. Eur. |
| Heavy Metals | Cd | Max. 0.1 mg/kg | <0.1 | mg/kg | accredited |
| | Hg | Max. 0.1 mg/kg | <0.1 | mg/kg | external |
| | Pb | Max. 0.1 mg/kg | <0.1 | mg/kg | laboratory |
| | As | Max. 0.1 mg/kg | <0.1 | mg/kg | |

| | | | | |
|---|---|---|---|---|
| PCBs (polychlorinated | NMT 0.5 ppm | <0.005 | ppm | accredited |

| | |
|---|---|
| biphenyls, IUPAC | external |
| congeners PCB-28, | laboratory |
| PCB-52, PCB-101, | |
| PCB-118, PCB-138, | |
| PCB-153, PCB-180) | |

### Omega-3 Fatty Acid Ethyl Ester Certificate of Analysis Continued

| | | | |
|---|---|---|---|
| Dioxins | The sum of PCDDs and | 0.51 pg/g WHO-TEQ | accredited external |
| | PCDFs is NMT 1 pg/g of | | laboratory |
| | WHO-TEQ | | |
| Σ Dioxins + | Max. 10.0 pg TEQ(WHO)/g | 1.82 pg TEQ(WHO)/g | accredited external |
| Dioxin-like PCBs | | | laboratory |

| Parameter | | Specification | Analysis | | Method |
|---|---|---|---|---|---|
| Fatty | EPA | NLT 430 mg/g | 458 | mg/g | GC |
| Acids | | NMT 495 mg/g | | | |
| (as EE) | DHA | NLT 347 mg/g | 365 | mg/g | |
| | | NMT 403 mg/g | | | |
| | ≥EPA+DHA | NLT 800 mg/g | 823 | mg/g | |
| | | NMT 880 mg/g | | | |
| | Total Omega-3 | NLT 90% (w/w) | 91% | (w/w) | |
| | Total Omega-6 | NMT 45 mg/g | 27 | mg/g | |
| | SDA EE C18:4 n-3 | 4.0-37.0 mg/g | 14.8 | mg/g | |
| | HPA EE C21:5n-3 | 7.9-31.4 mg/g | 21.7 | mg/g | |
| | DPA EE C22:5 n-3 | 16.3-50.0 mg/g | 38.9 | mg/g | |
| | ETA EE C20:4 n-3 | 1-20 mg/g | 8 | mg/g | |

| | | |
|---|---|---|
| ALA EE C18:3n-3 NMT 2.0 mg/g | 1.0 | mg/g |

| Limit of Total Unidentified | The area of the largest single | <0.5% | | GC |
|---|---|---|---|---|
| Fatty Acid Ethyl Esters | unidentified peak is NMT 0.5% of the | | | |
| | total area. | | | |
| | The total area of unidentified peaks as | <2% | | |
| | calculated above is NMT 2%. | | | |
| Previously unidentified fatty | | <0.5% | | GC |
| acid peaks now identified as | | each | | |
| - trans fatty acids | NMT 0.5% each | | | |
| - conjugated dienes | NMT 0.5% each | <0.5% | | |
| | | each | | |
| - Σ trans fatty acids + | NMT 2% | <2% total | | |
| conjugated dienes | | | | |
| Oligomers | NMT 1.0% | 1.0% | | USP/Ph. Eur. |
| Alpha-Tocopherol, NF | 0.36% - 0.44% | 0.41% | | |
| Cholesterol | NMT 3.0 mg/g | 1.2 | mg/g | |

NMT = not more than; NLT = not less than

### Omega-3 Fatty Acid Ethyl Ester Certificate of Analysis (KD Pharma) Continued

| Parameter | | Specification | Analysis | | Method |
|---|---|---|---|---|---|
| Pesticides | DDE (dichlorodiphenyl- | NMT 50 ppb | <16 | ppb | accredited external |
| | dichloroethylene) | | | | laboratory |
| | DDT (dichlorodiphenyl- | NMT 50 ppb | <16 | ppb | |
| | tricholoroethane) | | | | |
| | HCB | NMT 50 ppb | <40 | ppb | |
| | (Hexachlorobenzene) | | | | |
| | Lindane | NMT 50 ppb | <40 | ppb | |
| BFR (PBDEs)* | | NMT 500 ppb | <500 | ppb | accredited external |
| | | | | | laboratory |
| Vitamin A* | | NMT 10 ppm | <10 | ppm | accredited external |
| | | | | | laboratory |
| Vitamin D3* | | NMT 10 IU/g | <10 | IU/g | accredited external |
| | | | | | laboratory |
| Water* | | NMT 0.1% | <0.1% | | accredited external |
| | | | | | laboratory |
| Se | | NMT 0.1 ppm | <0.1 | ppm | accredited external |
| | | | | | laboratory |
| Cu | | NMT 0.3 ppm | <0.3 | ppm | accredited external |
| | | | | | laboratory |
| Fe | | NMT 14 ppm | <14 | ppm | accredited external |
| | | | | | laboratory |
| Unsaponifiable matter* | | NMT 1.5% | <1.5% | | Optionally at accredited |
| | | | | | external laboratory |
| Microbial | Total Aerobic | NMT 1000 | <1000 | CFU/g | accredited external |
| Limits* | Microbial Count | CFU/g | | | laboratory |
| | (TAMC) | | | | |
| | Total Combinated | NMT 100 CFU/g | <100 | CFU/g | accredited external |
| | Yeasts/Moulds Count | | | | laboratory |

| | | | |
|---|---|---|---|
| (TYMC) | | | |
| E. coli | Absent/g | Absent | accredited external |
| | | | laboratory |
| Staphylococcus aureus | Absent/g | Absent | accredited external |
| | | | laboratory |
| Salmonella | Absent/10 g | Absent | accredited external |
| | | | laboratory |
| Enterobacteria (bile- | Absent/g | Absent | accredited external |
| tolerant gram-negative | | | laboratory |
| bacteria) | | | |

*Skip lot tested - at least one batch tested per year

The EPA n-3 EE concentration was determined to be approximately 2,000 mg/10 mL and the DHA n-3 EE concentration was approximately 1,600 mg/10 mL, as determined by gas chromatography.

The oral solution content contains at least 3,024 mg of the combined amount of EPA n-3 EE and DHA n-3 EE.

The oral solution content contains at least 3,600 mg of total omega-3 fatty acid ethyl esters.

The five minor Omega-3 ethyl esters comprise approximately 400 mg per day, and the amount of total Omega-3 ethyl esters is 4,000 mg per day.

An oral composition of the present disclosure is exemplified in Table 1.

TABLE 1
Omega-3-acid EE Oral Solution Composition

| Component | mg/10 mL | | grams/10 mL | |
|---|---|---|---|---|
| Omega-3-Acid Ethyl Esters | 4000.0 | | 4.00 | |
| L-Ascorbyl Palmitate, NF | 8.0 | | 0.008 | |
| Alpha-Tocopherol, USP | 20.4 | | 0.0204 | |
| Lecithin | 20.0 | | 0.02 | |
| Methylparaben, NF | 5.0 | | 0.005 | |
| Propylparaben, NF | 1.0 | | 0.001 | |
| Saccharin, NF | 3.5 | | 0.0035 | |
| Lemon Mint Flavor | 56.0 | | 0.056 | |
| Lemon Flavor | 200.0 | | 0.20 | |
| Optify ® Masking Flavor | 27.2 | | 0.0272 | |
| Refined Olive Oil (grade IV) | 4758.9 | | 4.7589 | |
| Total Weight | 9100 | mg | 9.1 | grams |
| Total Volume | 10.0 | mL | 10.0 | mL |

Assay values for the Omega-3 EE Oral Solution are shown in Table 2.

TABLE 2
Quantitative Results for Omega-3-acid Ethyl Esters Oral Solution

| | mg/10 mL dose * |
|---|---|
| Omega-3 Oral Solution | |
| EPA n-3 EE | 2012.8 |
| DHA n-3 EE | 1611.0 |
| Σ (EPA n-3 EE + DHA n-3 EE) | 3623.8 |
| Ethyl Esters | |
| Σ (five minor n-3 EE) | 398.3 |
| Omega-3 EE | |
| Total | 4022.1 |

* Dose for omega-3-acid ethyl esters oral solution (oral pharmaceutical composition) was 10 mL per day. Target omega-3 ethyl ester content = 4000 mg/10 mL dose

### Example 3. Characterization of Lovaza™ Capsules

Lovaza® (omega-3-acid ethyl ester capsules) is indicated as an adjunct to diet to reduce triglyceride levels in adult patients with severe hypertriglyceridemia (greater than or equal to 500 mg/dL).

Lovaza®, a lipid-regulating agent, is supplied as a liquid-filled gel capsule for oral administration. Each 1-gram capsule of Lovaza™ (omega-3-acid ethyl esters) contains at least 900 mg of the ethyl esters of omega-3 fatty acids. These are predominantly a combination of ethyl esters of eicosapentaenoic acid (EPA-approximately 465 mg) and docosahexaenoic acid (DHA-approximately 375 mg).

The prescribed dosage of Lovaza® is four capsules per day (QID). This may be implemented as four capsules taken one time a day, or as two capsules taken in the morning and two capsules taken in the evening.

The amount of the EPA ethyl ester and the DHA ethyl ester is 3,360 mg per day.

Lovaza™ capsules contain a minimum daily dose of 3,600 mg total Omega-3 ethyl esters.

TABLE 3
Quantitative Values for Lovaza ™ Capsules

| | mg/capsule | mg/4 capsule dose ** |
|---|---|---|
| Lovaza ™ Capsules | | |
| EPA n-3 ee | 440.7 | 1762.8 |
| DHA n-3 ee | 377.0 | 1508.0 |
| Σ (EPA n-3 ee + DHA n-3 ee) | 817.7 | 3270.8 |
| Ethyl Esters | | |
| Σ (five minor n-3 ee) | 79.8 | 319.2 |
| Omega-3 ee | | |
| Total | 897.5 | 3590.0 |

** Lovaza ™ dose is 4 capsules per day; minimum omega-3 ethyl ester content = 3,600 mg/4 capsule dose.

### Example 4. Characterization of NatureMade™ OTC Supplement Capsule

Nature Made® is an over-the-counter (OTC) omega-3 product. Nature Made® markets omega-3 products in a variety of delivery systems (capsules, gummies), and chemical entities (triglyceride (TG), and ethyl ester (EE) forms). The free fatty acid (FFA) form also exists among other suppliers within the OTC marketplace. The specific formulation evaluated herein was the Nature Made® omega-3 (ethyl esters) from fish oil, supplied as a liquid-filled gel capsule for oral administration. The concentration of total omega-3-acid ethyl esters is 720 mg per Nature Made® capsule.

Each capsule of Nature Made® (omega-3-acid ethyl esters) contains 1.2 grams of fish oil concentrate. This concentrate contains at least 720 mg of the ethyl esters of omega-3 fatty acid. These are predominantly a combination of EPA n-3 EE and DHA n-3 EE.

The EPA n-3 EE concentration is approximately 360 mg/capsule and the DHA n-3 EE concentration is approximately 300 mg/capsule. This is equivalent to a combined EPA ethyl ester and DHA ethyl ester content of 660 mg per capsule.

The five minor omega-3 ethyl esters comprise 60 mg per day, and the amount of total omega-3-acid ethyl esters is 720 mg per day.

TABLE 4
Quantitative Results for Nature Made ® OTC Capsules

| | mg/capsule | mg/dose *** |
|---|---|---|
| Nature Made ® Capsules | | |
| EPA n-3 EE | 390.8 | 390.8 |
| DHA n-3 EE | 337.8 | 337.8 |
| Σ (EPA n-3 EE + DHA n-3 EE) | 728.6 | 728.6 |
| Ethyl Esters | | |
| Σ (five minor n-3 EE) | 123.4 | 123.4 |
| Omega-3 | | |
| Total | 852.0 | 852.0 |

*** Dose for Nature Made ® is 1 capsule per day
Target omega-3 ethyl ester content = 720 mg/dose

### Example 5. Comparison of Omega-3 EE Oral Solution Vs. Prescription Capsules Vs. OTC Supplement

Table 5 outlines some of the differences with respect to dosing and health benefits between the three comparator products.

TABLE 5
Comparison of LOVAZA ®, Nature Made ® OTC Supplements,
and Omega-3-acid Ethyl Esters Oral Solution

| Product | Characteristics |
|---|---|
| LOVAZA ® | Regulated by the FDA for purity and dosing |
| | standards. |
| | Doses of 2 capsules twice daily or 4 capsules |
| | once daily, provides 4 grams of omega-3 per day, a |
| | dose demonstrated to lower triglyceride levels. |
| | FDA-approved for the treatment of |
| | hypertriglyceridemia. Reference: LOVAZA ® |
| | website |
| Nature Made ® | Does not contain a therapeutically significant |
| | amount of Omega-3 EPA or DHA. |
| | Not indicated to treat heart disease. |
| | Can contain oils other than omega-3, |
| | responsible for putrefaction (fishy smell, fishy |
| | eructation (burps)). |
| Omega-3-acid | Convenience of single oral dose versus |
| Ethyl Ester Oral | multiple large capsules |
| Solution of the | Regulated by the FDA for purity and dosing |
| present disclosure | standards. |
| | Single 10 mL daily dose daily, provides 4 |
| | grams of omega-3 per day, a dose demonstrated to |
| | lower triglyceride levels. |
| | Meets FDA and USP concentrations approved |
| | for the treatment of hypertriglyceridemia. |

### Example 6. Comparison of EPA EE and DHA EE in Product Formulation

Table 6 compares the assay content (as determined by gas chromatography) of EPA n-3 EE and DHA n-3 EE across the three products in Table 5.

TABLE 6
EPA n-3 EE and DHA n-3 EE Content Assay

| Omega-3 Oral Solution | mg/10 mL dose |
|---|---|
| EPA n-3 EE | 2012.8 |
| DHA n-3 EE | 1611.0 |
| Σ (EPA n-3 EE + DHA n-3 EE) | 3623.8 |

| Lovaza ® Capsules | mg/4 capsule dose |
|---|---|
| EPA n-3 EE | 1762.8 |
| DHA n-3 EE | 1508.0 |
| Σ (EPA n-3 EE + DHA n-3 EE) | 3270.8 |
| Nature Made ® OTC Capsules | mg/1 capsule dose |
| EPA n-3 EE | 390.8 |
| DHA n-3 EE | 337.8 |
| Σ (EPA n-3 EE + DHA n-3 EE) | 728.6 |

### Example 7. Comparison of Σ Five Minor Ethyl Esters in Formulation

Total omega-3-acid ethyl esters are calculated according to the following equation:

Total Omega-3Ethyl Esters=EPA n-3EE+DPA n-3EE+Σfive minor n-3ethyl esters

Tables 7 and 8 describe the Σ Five Minor n-3 Ethyl Esters and present the percent assay across the three products.

The five minor ethyl esters, as designated by USP, are shown in Table 7.

TABLE 7
Five minor ethyl ester compounds

| Abbreviation | Full Name | Formula |
|---|---|---|
| ALA n-3 EEEE | Alpha-linolenic acid ethyl ester | C₂₀H₃₄O₂ |
| SDA n-3 EE | Stearidonic (moroctic) acid ethyl ester | C₂₀H₃₂O₂ |
| ETA n-3 EE | Eicosatetraenoic acid ethyl ester | C₂₂H₃₆O₂ |
| HPA n-3 EE | Heneicosapentaenoic acid ethyl ester | C₂₃H₃₆O₂ |
| DPA n-3 EE | Docosapentaenoic acid ethyl ester | C₂₄H₃₈O₂ |

Table 8 compares the percent of the Σ five minor ethyl esters (as determined by gas chromatography), relative to the total of all Omega-3-acid ethyl esters present among Lovaza®, Nature Made®, and the oral solution composition of the present disclosure.

TABLE 8
Percent of Σ Five Minor Ethyl Esters

| Product Formulation | Percent |
|---|---|
| Omega-3 ee Oral Solution | 9.9 |
| Lovaza ® Capsules | 8.9 |
| Nature Made ® OTC Supplement | 14.5 |

### Example 8. Comparison of Omega-6 Ethyl Esters

Omega-6 fatty acid compounds are believed to be inflammatory and may be counter-productive to the taking of omega-3 fatty acid therapy.

The oral composition of the present disclosure contains relatively less omega-6 concentration compared to both the Nature Made® OTC and Lovaza® capsules (Table 9) (as determined by gas chromatography (GC)).

TABLE 9
Quantitative Results of Omega-6 Compounds using GC

| | Percent |
|---|---|
| Omega-3 Oral Solution | |
| ETA n-6 EE | 0.8 |
| DPA n-6 EE | 0.7 |
| Total Omega n-6 EE | 1.5 |
| Lovaza ® Rx Capsules | |
| ETA n-6 EE | 2.2 |
| DPA n-6 EE | 1.0 |
| Total Omega n-6 EE | 3.2 |
| Nature Made ® OTC Capsules | |
| ETA n-6 EE | 2.1 |
| DPA n-6 EE | 0.5 |
| Total Omega n-6 EE | 2.6 |

### Example 9. Comparison of Other Ethyl Esters Across Formulations

Oral solution superior purity versus Nature Made® OTC capsules was quantitated (as determined by gas chromatography as other ethyl esters in Table 10.

TABLE 10
Quantitative Results of Other Ethyl Ester Compounds using GC

| | Percent |
|---|---|
| Omega-3 Oral Solution | |
| Other Identified EE | 1.3% |
| Other Unidentified EE | 2.8% |
| Other Total EE | 4.1% |
| Lovaza ® Rx Capsules | |
| Other Identified EE | 0.0% |
| Other Unidentified EE | 1.1% |
| Other Total EE | 1.1% |
| Nature Made ® OTC Capsules | |
| Other Identified EE | 10.3% |
| Other Unidentified EE | 15.9% |
| Other Total EE | 26.2% |

### Example 10. Antioxidant Experimentation

Primary oxidation processes in oil mainly form hydroperoxides, which are measured by the Peroxide Value (PV). In general, the lower the PV, the better the quality of the oil. However, PV decreases as secondary oxidation products appear. If oxidation is more advanced, the PV may be relatively low.

The secondary stage of oxidation occurs when the hyperoxides decompose to form carbonyls and other compounds, in particular, aldehydes. These are what gives the oil a rancid smell, and they are measured by the Anisidine Value (AV). The lower the AV, the better the quality of the oil.

Each of the flavoring components that were selected for the final formulation contain oxygen-containing chemical species, such as aldehydes, that contribute to the overall anisidine value.

### Example 11. Selection of Liquid Carrier

A summary of different liquid carriers considered for formulating the omega-3 fatty acid ethyl esters is provided below.

ALDO MO: Mono and diglycerides. Difficulties in dissolving Omega-3 API. Precipitation out of solution occurred in the form of small irregular-shaped masses.

Capmul GMO-50: Mono and diglycerides. Precipitation out of solution occurred in the form of small irregular-shaped masses.

Corn Oil: Corn oil was not chosen, as a result of high levels of the unhealthy Omega-6 fatty acid, linoleic acid.

Soybean Oil: Ascorbyl palmitate fell out of solution with soybean oil. Oily texture deemed unacceptable from a taste and palatability standpoint.

Peceol: Mono and diglycerides, glycerol monooleate. After sitting at room temperature for a few days, precipitation out of solution occurred in the form of small irregular-shaped masses.

Olive Oil (NF): The olive oil was NF grade supplied by Spectrum Chemical. Initial formulation improvement utilizing olive oil, however, solutions made with this grade of olive oil, degraded quickly.

Refined Olive Oil Grade IV: This highly refined grade of olive oil was developed by the supplier (Archer Daniels Midland) for use in the manufacture of pharmaceutical products.

### Grade IV Olive Oil Certificate of Analysis

| | | | | Specifications | |
|---|---|---|---|---|---|
| Characteristics | | | Results | EP/USP-NF | Method |
| Identification | | | complies | meets the | See test for Fatty |
| | | | | requirements of | acid composition |
| | | | | the test for fatty | |
| | | | | acid composition | |
| Acid Value | | < | 0.1 | NMT 0.3 (EP)- | EP 2.5.1 |
| | | | | 0.2 (USP) | |
| Peroxide value (mEq O₂/Kg) | | | 0.1 | NMT 5.0 | EP 2.5.5 Part |
| | | | | | A∫USP401 |
| Water content (%) | | ≤ | 0.1 | NMT 0.1 | EP 2.5.32/USP 921 |
| | | | | | Method IC |
| Unsaponifiable matter (%) | | | 0.8 | NMT 1.5 | EP olive oil |
| | | | | | monograph |
| Alkaline impurities (ml HCI | | ≤ | 0.1 | NMT 0.1 | EP 24.19/USP-NF |
| 0.01N/10 mL) | | | | | Olive oil |
| | | | | | monograph |
| Sesame Oil | | | complies | No bluish green | EP/USP-NF Olive |
| | | | | color | oil monograph |
| Specific absorbance at 270 nm | | | 0.21 | NMT 1.20 | EP/USP-NF Olive |
| | | | | | oil monograph |
| Fatty acid composition (%) | | | | | |
| Saturated fatty | <C 16:0 | ≤ | 0.1 | NMT 0.1 | Internal method |
| acids | | | | | validated vs EP |
| | | | | | 24.22 Part A/USP |
| | | | | | 401 |
| Palmitic acid | C 16:0 | | 9.7 | 7.5-20.0 | |
| Palmitoleic | C 16:1 | | 0.6 | NMT 3.5 | |
| acid | | | | | |
| Stearic acid | C 18:0 | | 3.2 | 0.5-5.0 | |
| Oleic acid* | C 18:1 | | 80.2 | 56.0-85.0 | |
| Linoleic acid | C 18:2 | | 4.7 | 3.5-20.0 | |
| Linolenic acid | C 18:3 | | 0.6 | NMT 1.2 | |
| Arachidic acid | C 20:0 | | 0.4 | NMT 0.7 | |
| Eicosenoic | C 20:1 | | 0.3 | NMT 0.4 | |
| acid | | | | | |
| Behenic acid | C 22:0 | ≤ | 0.1 | NMT 0.2 | |
| Lignoceric | C 24:0 | ≤ | 0.1 | NMT 0.2 | |
| acid | | | | | |
| Sterol Composition (%) | | | | | |
| Cholesterol | | ≤ | 0.3 | NMT 0.5 | EP 2.4.23 Part |
| | | | | | B/USP-NF Olive |
| | | | | | oil monograph |
| Campesterol | | | 3.2 | NMT 4.0 | |
| Δ7-stigmasterol | | ≤ | 0.3 | NMT 0.5 | |
| Sum of β-sitosterol, Δ5,23- | | | 95.7 | NLT 93.0 | |
| stigmastadienol, clerosterol, | | | | | |
| sitostanol, Δ5-avenasterol, | | | | | |
| Δ5,24-stigmastadienol | | | | | |
| Stigmasterol ≤ Campesterol | | | complies | complies | |
| Additional data | | | | | |
| Bacterial | (IU/g or | ≤ | | NMT 1.25 | |
| endotoxins | EU/g) | | | | |

*and isomer

Olive oil is resistant to heat degradation. The batch was heated to 60 to 100 °C to dissolve all ingredients.

TABLE 11
Anisidine Values for Stability Solutions at 25 °C

| Solution Spike | Initial | 1 month | 2 month | 3 month |
|---|---|---|---|---|
| Olive Oil, NF | 6.638 | 12.40 | 29.70 | 35.89 |
| Olive Oil, NF and | 7.135 | 6.67 | 14.79 | 14.26 |
| Ascorbyl Palmitate | | | | |

The presence of lecithin and ascorbyl palmitate led to a more stable formulation (lower anisidine values) than when compared to a matrix containing ascorbyl palmitate, but with no lecithin present.

TABLE 12
Anisidine Values for Stability Solutions at 40 °C

| Solution Spike | Initial | 1 month | 2 month | 3 month |
|---|---|---|---|---|
| Ascorbyl Palmitate | 13.19 | 15.12 | 17.07 | 17.33 |
| Ascorbyl Palmitate | 13.00 | 11.24 | 8.75 | 10.29 |
| and Lecithin | | | | |

### Example 12. Stability Studies

### HDPE Brown Bottle:

Stability Study initiated with Omega-3 EE oral solution stored in HDPE brown bottles. The bottle collapsed during stability storage at elevated temperature. Two-week stability results for EPA n-3 EE+DHA n-3 EE assay were out-of-specification high with olive oil loss (Table 13). The HDPE brown bottle study was discontinued.

TABLE 13
EPA n-3 EE + DHA n-3 EE Stability Results

| Sample | Condition | Initial | 2 weeks | 1 month |
|---|---|---|---|---|
| 084 | 2 °C-8 °C | 102.9 | 109.2 | 109.1 |
| 084 | 25 °C/60% RH | 102.9 | 110.3 | 109.1 |
| 084 | 40 °C/75% RH | 102.9 | 114.1 | 110.7 |

### PET Amber Bottle:

Polyethylene terephthalate (PET) bottle performed better than HDPE bottles. Glass TOC vial, 6 ounce HDPE, 8 ounce HDPE, and 12 ounce PET bottles were compared.

HDPE Bottles: Appearance reported as bowed (collapsed) bottle.

HDPE Bottles: Taste reported as Very Fishy at 1 month.

PET Bottle: Taste reported as Not Fishy at 1 month.

Polyethylene terephthalate (PET) bottle exhibited lower anisidine and peroxide values than did HDPE bottles. PET bottles were also resistant to olive oil permeation.

### Example 13. Registration Batches

Registration Batches: Batches 2302635, 2302637, and 2302672, all prepared in accordance with the ingredients and amounts in Example 2 (Table 1) were packaged in the following container-closure system.

Bottle: ALPHA packaging, 24-410 Modern round dark amber polyethylene terephthalate (PET), 12 ounce bottle (300 mL fill).

Closure: 24-400 CRC white polypropylene cap, pulp backed heat induction liner.

The three batches were placed upon stability testing. For this stability study, each of the three stability batches were stored in both upright and horizontal positions within the appropriate stability chambers.

Samples were stored under the following conditions:
25 °C±2 °C/60% RH±5% RH for 24 months
30 °C±2 °C/65% RH±5% RH for 12 months
40 °C±2 °C/75% RH±5% RH (accelerated stability for 6 months)

Stability testing was performed over twenty-four months. The specifications for the oral solutions are listed in Tables 16-17 below.

### Appearance/Description

Samples of the three registration batches, for both upright and horizontal storage conditions and for all storage conditions, were assessed for their appearance. A clear, yellow solution that was free of particles with a citrus aroma was noted for all samples. Analyses for Description (Visual Description) were within specification at all test intervals at all conditions and at both orientations.

### Weight Loss

Weight loss of samples of the three registration batches, for both upright and horizontal storage conditions and for all storage conditions, were assessed.

All data conformed to the product specification of “NMT 1 of the 10 test containers exceeds 2.5%/year percentage of weight loss and does not exceed 5.0%/year in any of them”.

TABLE 14
Weight Loss Rate Data

| Time | Storage | 2302635 | | 2302637 | | 2302672 | |
|---|---|---|---|---|---|---|---|
| (months) | Condition | Upright | Horizontal | Upright | Horizontal | Upright | Horizontal |
| 3 | 25 °C | 0.4 | 0.4 | 0.4 | 0.4 | 0.4 | 0.4 |
| 6 | 25 °C | 0.2 | 0.2 | 0.2 | 0.2 | 0.2 | 0.2 |
| 9 | 25 °C | 0.1 | 0.1 | 0.2 | 0.2 | 0.2 | 0.2 |
| 12 | 25 °C | 0.1 | 0.1 | 0.1 | 0.1 | 0.1 | 0.1 |
| 18 | 25 °C | 0.1 | 0.1 | 0.1 | 0.1 | 0.1 | 0.1 |
| 24 | 25 °C | 0.1 | 0.1 | 0.1 | 0.1 | 0.1 | 0.1 |
| 3 | 30 °C | 0.5 | 0.5 | 0.5 | 0.5 | 0.5 | 0.5 |
| 6 | 30 °C | 0.3 | 0.3 | 0.3 | 0.3 | 0.3 | 0.3 |
| 9 | 30 °C | 0.2 | 0.2 | 0.2 | 0.2 | 0.2 | 0.2 |
| 12 | 30 °C | 0.2 | 0.2 | 0.2 | 0.2 | 0.2 | 0.2 |
| 1 | 40 °C | 1.8 | 1.8 | 1.9 | 1.9 | 1.9 | 1.9 |
| 2 | 40 °C | 1.1 | 1.2 | 1.2 | 1.2 | 1.2 | 1.2 |
| 3 | 40 °C | 0.8 | 0.8 | 0.8 | 0.8 | 0.8 | 0.8 |
| 6 | 40 °C | 0.5 | 0.5 | 0.5 | 0.5 | 0.5 | 0.5 |

### Color Analysis

The color of samples of the three registration batches, for both upright and horizontal storage conditions and for all storage conditions, was assessed using UV-VIS spectrophotometry at 400 nm.

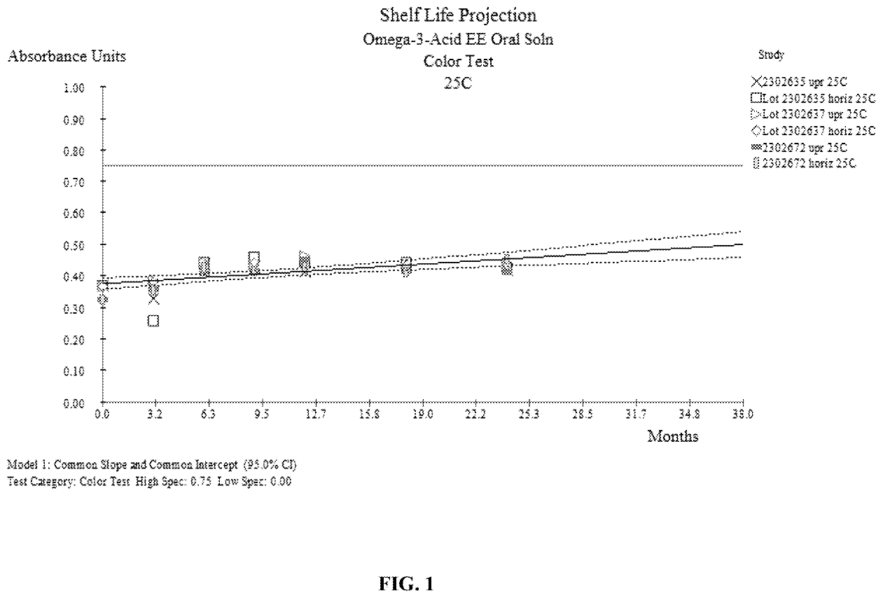

The color analysis results ranged from 0.2588-0.4633 for the 25 °C samples (FIG. 1), 0.3266-0.4763 for the 30 °C samples, and 0.3266-0.4828 for the 40 °C samples. All results were within the specification of NMT 0.75.

### Specific Gravity

Samples of the three registration batches, for both upright and horizontal storage conditions and for all storage conditions, were assessed for specific gravity. The specific gravity remained at 0.91 for all lots at all test stations and conditions. This was within the specification of 0.89-0.93.

### Acid Value

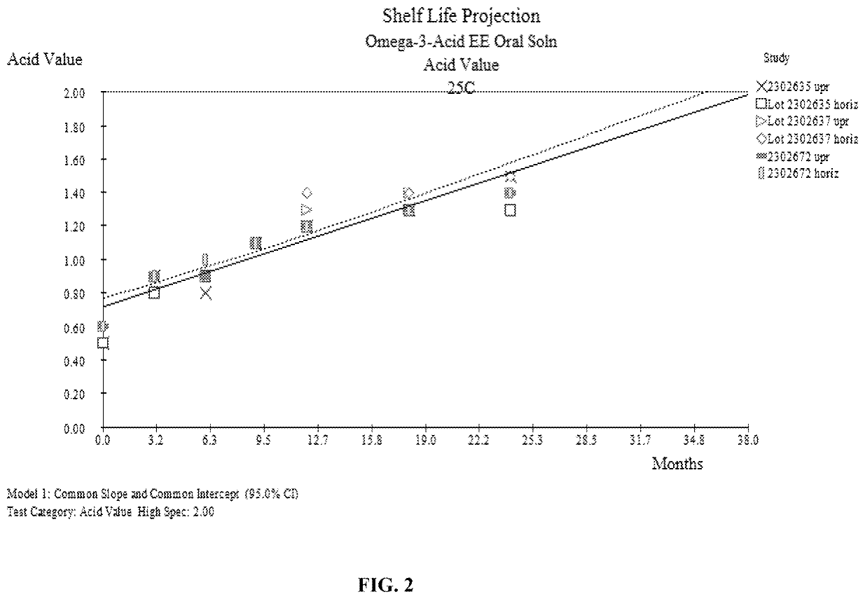

The acid value was measured for the samples of the three registration batches, for both upright and horizontal storage conditions and for all storage conditions. The acid value results ranged from 0.5-1.5 for samples at all conditions. The results conformed to the specification of NMT 2.0. The room temperature condition data for the Acid Value is illustrated in FIG. 2.

### Anisidine and Peroxide Values

Samples of the three registration batches, for both upright and horizontal storage conditions and for all storage conditions, were assessed for Peroxide and Anisidine Values.

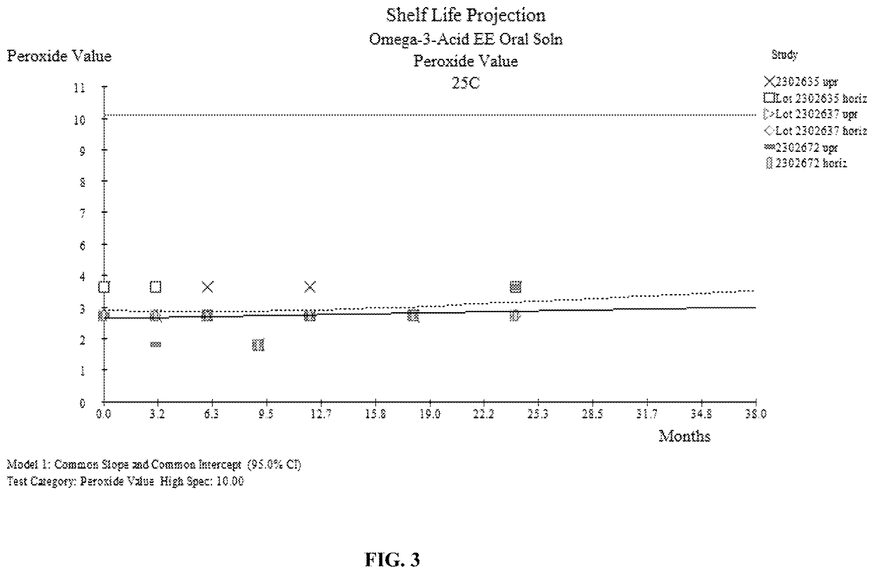

The Peroxide Value (PV) for the registration batches ranged from 2-4 for the samples at all conditions. These values were below the PV specification of NMT 10 meq/kg. The room temperature condition data for the PV is illustrated in FIG. 3.

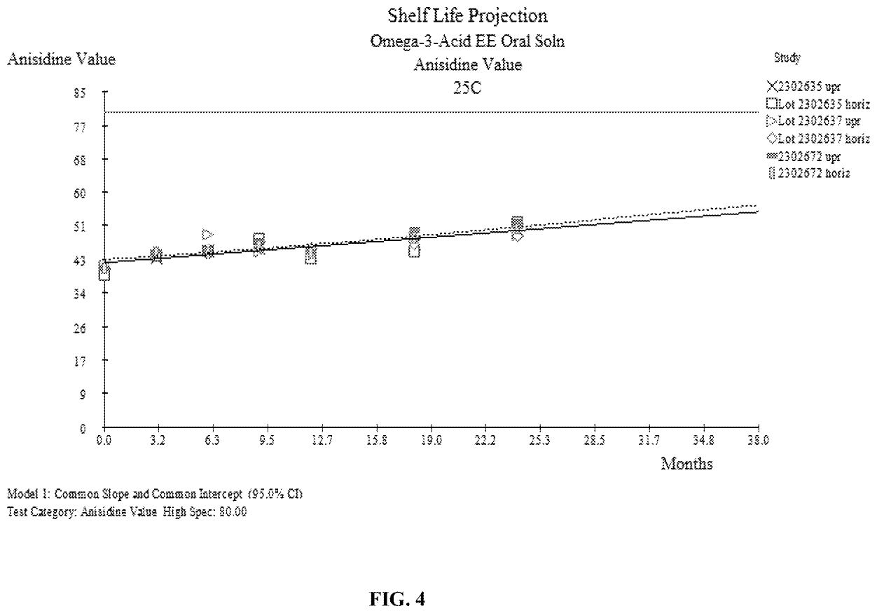

The Anisidine Value (AV) for the registration batches ranged from 38.6-52.0 for the 25 °C samples, 38.6-48.4 for the 30 °C samples, and 38.6-48.5 for the 40 °C samples. These values were below the AV specification of NMT 80. The room temperature condition data for the AV is illustrated in FIG. 4.

### Parabens

Samples of the three registration batches, for both upright and horizontal storage conditions and for all storage conditions, were assessed for their paraben content.

The methylparaben content ranged from 97.5%-99.2% for the 25 °C samples, 97.6%-98.5% for the 30 °C samples, and 96.4%-98.5% for the 40 °C samples. Analyses for methylparaben were within the specification of 90.0%-110.0% at all test intervals and conditions.

The propylparaben content ranged from 95.4%-99.3% for the 25 °C samples, 95.4%-100.4% for the 30 °C samples, and 94.2%-99.6% for the 40 °C samples. Analyses for propylparaben were within the specification of 90.0%-110.0% at all test intervals and conditions.

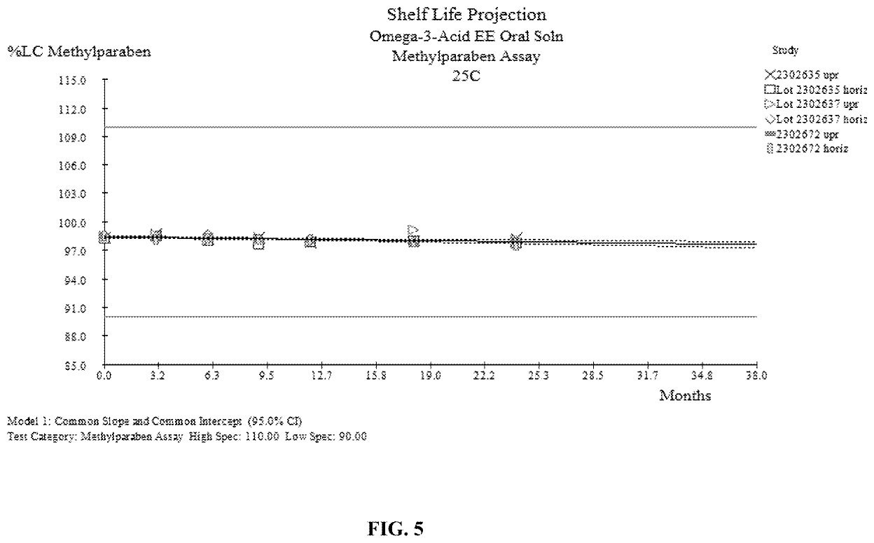

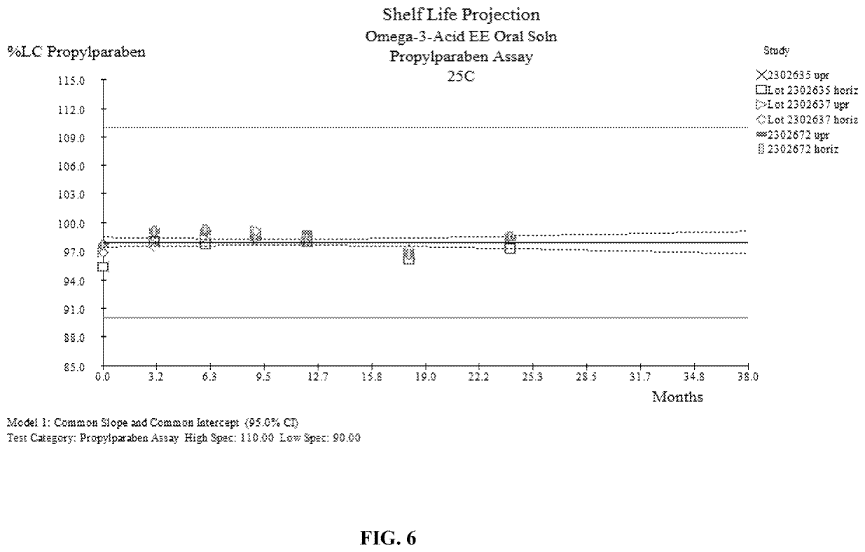

The room temperature paraben data is illustrated in FIG. 5 (methylparaben) and FIG. 6 (propylparaben).

### Alpha-Tocopherol

Alpha-tocopherol was added to the formulation as an antioxidant, and the alpha-tocopherol content for the samples of the three registration batches, for both upright and horizontal storage conditions and for all storage conditions, was measured.

The alpha-tocopherol content remained stable and ranged from 0.39%-0.42% for the 25 °C samples, 0.39%-0.42% for the 30 °C samples, and 0.40%-0.43% for the 40 °C samples. Analyses for alpha-tocopherol were well within the specification of 0.2%-0.5% at all test intervals and conditions.

### Omega-3-Acid Ethyl Esters

The Omega-3-Acid Ethyl Esters content remained well within specifications as shown in Table 15.

TABLE 15
Omega-3-Acid Ethyl Esters Content

| | | | | | EPAee + | | Total Omega- | | Sum of 5 minor | |
|---|---|---|---|---|---|---|---|---|---|---|
| | EPAee | | DHAee | | DHAee | | 3-Acid EE | | EE | |
| Condition | Min | Max | Min | Max | Min | Max | Min | Max | Min | Max |
| 25 °C | 1975 | 2141 | 1495 | 1674 | 3476 | 3814 | 3890 | 4539 | 414 | 736 |
| 30 °C | 2053 | 2156 | 1508 | 1685 | 3575 | 3841 | 4163 | 4582 | 569 | 791 |
| 40 °C | 2056 | 2159 | 1524 | 1716 | 3603 | 3875 | 4202 | 4519 | 584 | 736 |

| | | | | | | |
|---|---|---|---|---|---|---|
| Specifications | Report Results | Report Results | 3024 | 4000 | NLT 3600 | Report Results |

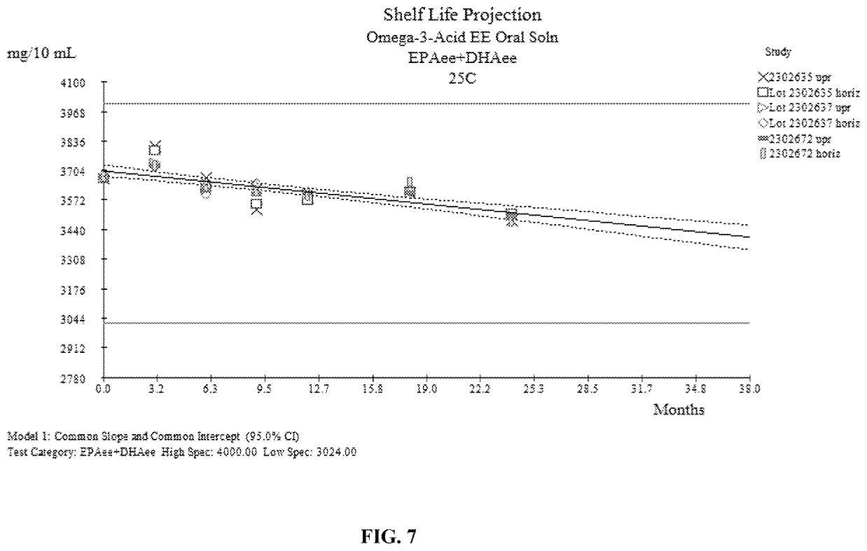

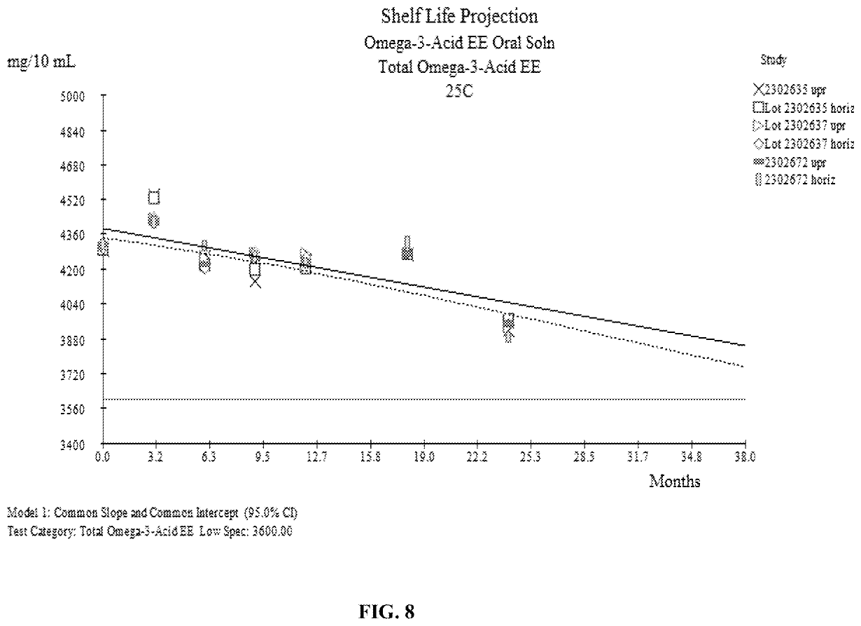

The room temperature DHA+EPA Ethyl Esters data is graphed in FIG. 7. The room temperature Total Omega-3-Acid Ethyl Esters data is graphed in FIG. 8.

The assay values for the three registration batches, for both upright and horizontal storage conditions, were calculated. These results are shown in FIG. 7. The results for the EPA n-3 EE+DHA n-3 EE assay fully support expiration dating through 24 months.

Similarly, the total omega-3 ethyl esters values for the three registration batches, for both upright and horizontal storage conditions, were calculated. These results are shown in FIG. 8. In addition, the results for the total omega-3 ethyl esters fully support expiration dating through 24 months.

For the registration batches, all the test conditions met the specifications for the assay of EPA n-3 EE+DHA n-3 EE, as well as for the assay of omega-3 ethyl esters.

### Microbial Limits

Microbial Testing per USP <60>, <61>, and <62>was performed on the lots at the relevant test intervals. The results conformed to USP requirements. The finished products were tested at the initial, twelve months, and 24 months test stations for the room temperature samples and at 12 months for the intermediate samples.

### Antimicrobial Effectiveness Testing (AET)

AET per USP <51>was performed on the lots at the relevant test intervals. The results conformed to USP requirements. The finished products were tested at the initial, twelve months, and 24 months test stations for the room temperature samples and at 12 months for the intermediate samples.

The chemistry specifications for Omega-3-acid EE Oral Solution are shown in Tables 16 and 17.

TABLE 16
Omega-3-acid Ethyl Ester Oral Solution Specifications

| Test | Specification |
|---|---|
| Appearance | Clear, yellow solution, free of particles |
| | with a citrus aroma. |
| Color analysis | NMT 0.75 |
| Identification A | The retention times of the peaks for |
| | eicosapentaenoic acid ethyl ester and |
| | docosahexaenoic acid ethyl ester of the sample |
| | solution correspond to those of the standard |
| | solution, as obtained in the determination of |
| | EPAee and DHAee. |
| Identification B | It complies with the specifications in the tests |
| | determination of EPAee and DHAee and |
| | Determination of total Omega-3-Acid Ethyl |
| | Esters. |
| EPA EE | Report EPA EE result (mg/10 mL) |
| DHA EE | Report DHA EE result (mg/10 mL) |
| EPA EE + DHA EE | Report EPA EE + DHA EE |
| Assay | Minimum: 3024 mg/10 mL |
| | Maximum: 4000 mg/10 mL |
| Total Omega-3 EE Assay | NLT 3600 mg/10 mL |

TABLE 17
Omega-3-acid Ethyl Ester Oral Solution Specifications

| Test | Specification |
|---|---|
| Acid Value | NMT 2.0 |
| Anisidine Value | NMT 80 |
| Peroxide Value | NMT 10 mEq/kg |
| Methylparaben Assay | 90.0-110.0% |
| Propylparaben Assay | 90.0-110.0% |
| Alpha Tocopherol Assay | 0.2-0.5% |
| Deliverable Volume | Stage One: The average volume from 10 |
| | containers is NLT 100% of the labelled |
| | volume, and no container is less than |
| | 95% of the labelled volume. |
| | Stage Two: The average volume from 30 |
| | containers is NLT than 100% of the labelled |
| | volume, and the volume from NMT 1 of 30 |
| | containers is less than 95% of the labelled |
| | volume, but NLT 90% of the labelled volume. |
| Weight Loss | NMT 1 of the 10 test containers exceeds |
| (Registration | 2.5%/year percentage of weight loss and |
| Batches Only) | does not exceed 5.0%/year in any of them |
| Specific Gravity | 0.89-0.93 |

TABLE 18
Microbial Specifications for Omega-
3-acid Ethyl Ester Oral Solution

| Test | Specification |
|---|---|
| Microbial Limits | Total Aerobic Microbial Count: |
| USP <60>, <61>, | Not more than 1000 cfu/g |
| USP <62> | Total Combined Yeast and Molds Count: |
| | Not more than 100 cfu/g |
| | Absence of Escherichia coli (1 g) |
| | Absence of Salmonella species (10 g) |
| | Absence of Staphylococcus aureus (1 g) |
| | Absence of Enterobacteria (1 g) |
| Antimicrobial | The product meets the requirements for category |
| Effectiveness | 3 type product: |
| Test USP <51> | Bacteria: NLT 1.0 log reduction from the initial |
| | count at 14 days, and no increase from the 14 |
| | days count at 28 days. |
| | Yeast and Molds: No increase from the initial |
| | calculated count at 14 and 28 days. |

### Conclusions

The results for the batches show that the Omega-3-Acid Ethyl Esters Oral Solution, 4g/10 mL, is a stable product. The methylparaben and propylparaben remained within specification, to ensure microbial contamination is prevented or minimized. The key indicators of degradation, including the appearance, acid value, anisidine and peroxide values, alpha-tocopherol, and omega-3 acid fatty acid ethyl ester content, demonstrate that the formulation was protected against oxidation.

The container closure system prevents the permeation of oxygen into the product, while the antioxidant system, comprising alpha-tocopherol, lecithin, and ascorbyl palmitate, prevents oxidation of the finished product.

### Example 14. Degradation Studies

### Conical Tube Preparation

Oral solution was transferred from an Omega-3-acid Ethyl Ester 16 ounce bottle containing finished product. Approximately 20 mL of drug product (oral solution) was transferred to a labelled 40 ml conical tube.

Content from the capsule products was removed carefully utilizing an 18-gauge disposable hypodermic needle affixed to a Henke-Ject 10 mL Luer-Lock syringe. Approximately 20 mL of drug product (capsule contents) was transferred directly to a 40 mL conical tube.

Plastic pipettes (VWR Part Number: 414004-001) were attached to the ends of the supply tubing leading from the air pump. Each tip was installed through a perforation cut through the Teflon/silicone septum of the centrifuge tube. Each conical tube was then loosely closed with the modified cap design to minimize the amount of aerosol (oil droplets) that could escape from the conical tube. A second perforation in the cap was also created to allow for excess air pressure to escape.

### Oxidation Procedure

Both the test product and the comparator product were oxidized concurrently. The air supply was furnished by a pump (TopFin Air-3000). The flow through each tip was measured at a rate of approximately 1000 mL/minute. Flow was maintained continuously (nights and weekends included) over the entire duration of the ten-day study.

At each of the designated timepoints, solution aliquots were taken from the conical tubes and transferred into individual 20 mL glass vials (VWR Part Number: 89093-854). The 20 mL vials were immediately sealed with the supplied vial caps and septa, and then were stored in the refrigerator until time of sample preparation.

The degradation rate over the course of the ten-day oxidation study was calculated for each test sample. The samples were evaluated for the presence of ethyl esters utilizing gas chromatography (GC). Analytical samples were then prepared according to the normal sample preparation and injected onto the GC system. The results are summarized in Table 19.

TABLE 19
Summary of Degradation Rates

| | | |
|---|---|---|
| | | EPA n-3 EE + |
| EPA n-3 EE | DHA n-3 EE | DHA n-3 EE |

Omega-3 Oral Solution

| | | |
|---|---|---|
| 0.0% | −1.1% | −0.5% |

Lovaza ® Rx Capsules

| | | |
|---|---|---|
| −4.2% | −5.5% | −4.8% |

Omega-3 Oral Solution

| | | |
|---|---|---|
| 0.2% | −1.6% | −0.6% |

Nature Made ® OTC Capsules

| | | |
|---|---|---|
| −6.7% | −10.5% | −8.4% |

The degradation rate observed for the Lovaza® capsule formulation was 9.6 times greater than the comparable oral solution. The degradation rate observed for the Nature Made® capsule formulation was 14.0 times greater than the comparable oral solution.

### Example 15. Olfactory Assessment Score

The test samples were subject to an olfactory assessment. A score based upon subjective evaluation of the intensity of any disagreeable odor was recorded.

An olfactory score ranking from 0 to 10 was recorded for each test station.

A score of 0 indicated no fishy odor was detected, and a score of 10 indicated a moderate degree of fishy odor was detected. A score of 10 was a sufficient degree of odor that would dissuade many users from not using the product.

This information is summarized in Table 20.

TABLE 20
Olfactory Score

| | Score |
|---|---|
| Nature Made ® OTC Capsules | |
| Day Zero | 1 |
| Day Three | 1 |
| Day Seven | 1 |
| Day Ten | 2 |
| Omega-3 Oral Solution | |
| Day Zero | 0 |
| Day Three | 1 |
| Day Seven | 1 |
| Day Ten | 0 |
| Comparison Solution | |
| Newly opened sachet | 10 |

The Omega-3-acid Ethyl Ester Oral Solution demonstrated improvement in olfactory assessment when compared to the Nature Made® OTC Supplement.

### Conclusion

The combination of a highly refined API, the utilization of olive oil (high purity) as diluent, the unique choice of antioxidants, and the choice of the container-closure system has the impact of creating a stable solution-based oral product.

1. An oral composition comprising:
a mixture of fatty acid esters comprising omega-3 fatty acid ethyl esters and omega-6 fatty acid ethyl esters; and
a liquid carrier comprising olive oil.

2.-39. (canceled)

40. A unit dose comprising the oral composition of claim 1 and having a volume from 1 mL to 20 mL.

41.-42. (canceled)

43. An oral composition comprising:
a mixture of fatty acid esters comprising omega-3 fatty acid ethyl esters and omega-6 fatty acid ethyl esters, wherein the oral composition comprises less than 6% omega-6 fatty acid ethyl esters relative to the omega-3 fatty acid ethyl esters; and
a liquid carrier.

44. The oral composition of claim 43, wherein the oral composition is in the form of a solution.

45. The oral composition of claim 43, wherein the oral composition comprises less than 2% ETA n-6 EE relative to the total omega-3 fatty acid ethyl esters.

46. The oral composition of claim 43, wherein the oral composition comprises less than 2% DPA n-6 EE relative to the total omega-3 fatty acid ethyl esters.

47. The oral composition of claim 43, wherein the mixture of fatty acid esters comprises at least 90% by weight of EPA n-3 EE, DHA n-3 EE, SDA n-3 EE, HPA n-3 EE, DPA n-3 EE, ETA n-3 EE, and ALA n-3 EE.

48. The oral composition of claim 43, wherein the oral composition comprises at least 1,500 mg total omega-3 fatty acid ethyl esters per 10 mL, wherein the total omega-3 fatty acid ethyl esters is the sum of EPA n-3 EE, DHA n-3 EE, SDA n-3 EE, HPA n-3 EE, DPA n-3 EE, ETA n-3 EE, and ALA n-3 EE).

49. The oral composition of claim 48, wherein the oral composition comprises from 1,500 mg to 5,000 mg total omega-3 fatty acid ethyl esters per 10 mL.

50. The oral composition of claim 43, wherein the oral composition comprises at least 1,500 mg of the sum of EPA EE and DHA EE per 10 mL.

51. The oral composition of claim 43, wherein the oral composition comprises from 1,500 mg to 5,000 mg total omega-3 fatty acid ethyl esters per 10 mL, wherein the sum of EPA n-3 EE and DHA n-3 EE is from 1,500 mg to 5,000 mg per 10 mL.

52.-53. (canceled)

54. The oral composition of claim 43, wherein the sum of EPA n-3 EE and DHA n-3 EE comprise at least 60% by weight of the total omega-3 fatty acid ethyl esters.

55. The oral composition of claim 43, wherein the ratio of EPA n-3 EE to DHA n-3 EE is from 5:1 to 1:5.

56. The oral composition of claim 43, wherein the liquid carrier comprises one or more non-aqueous liquids.

57. The oral composition of claim 56, wherein the one or more non-aqueous liquids is selected from olive oil, safflower oil, high-oleic safflower oil, sunflower oil, high-oleic sunflower oil, rapeseed oil, almond oil, avocado oil, sesame oil, soybean oil, corn oil, peanut oil, palm oil, cottonseed oil, flaxseed oil, coconut oil, grapeseed oil, hempseed oil, oleinate (monounsaturated fatty acid), canola oil, linseed oil, walnut oil, wheat germ oil, pumpkin seed oil, rice bran oil, and combinations thereof.

58. The oral composition of claim 56, wherein the one or more non-aqueous liquids comprises olive oil.

59. (canceled)

60. The oral composition of claim 43, wherein the oral composition further comprises at least one antioxidant, at least one preservative, at least one flavor ingredient, at least one sweetener, or a combination thereof.

61.-67. (canceled)

68. The oral composition of claim 43, wherein the oral composition does not comprise a preservative.

69.-76. (canceled)

77. The oral composition of claim 43, wherein the composition is in the form of a solution; the mixture of fatty acid esters comprises at least 90% by weight of EPA n-3 EE, DHA n-3 EE, SDA n-3 EE, HPA n-3 EE, DPA n-3 EE, ETA n-3 EE, and ALA n-3 EE; and the solution comprises, per 10 mL:
a. from 3,000 mg to 5,000 mg omega-3 fatty acid ethyl esters, wherein
i. the omega-3 fatty acid ethyl esters comprise EPA n-3 EE, DHA n-3 EE, SDA n-3 EE, HPA n-3 EE, DPA n-3 EE, ETA n-3 EE, and ALA n-3 EE;
ii. the sum of EPA n-3 EE and DHA n-3 EE represent from 2,000 mg to 5000 mg; and
iii, the weight ratio of EPA n-3 EE to DHA n-3 EE is from 2:1 to 1:1;
b. less than 6% omega-6 fatty ethyl esters relative to omega-3 fatty ethyl esters;
c. from 0.1 mg/10 mL to 250 mg/10 mL at least one antioxidant;
d. from 0.1 mg/10 mL to 1,000 mg/10 mL at least one flavor ingredient; and
e. from 0.1 mg/10 mL to 50 mg/10 mL at least one sweetener.

78. The oral composition of claim 77, wherein:
a. the at least one antioxidant is selected from ascorbyl palmitate, lecithin, alpha-tocopherol, and combinations thereof;
b. the at least one flavor ingredient is selected from lemon, a masking agent, mint, and a combination thereof;
c. the sweetener is saccharin; and
d. the olive oil is refined (grade IV) olive oil.

79. The oral composition of claim 43, wherein the oral composition is packaged in a polyethylene terephthalate (PET) bottle fitted with a closure.

80. (canceled)

81. A unit dose comprising the oral composition of claim 43 and having a volume from 1 mL to 20 mL.

82.-83. (canceled)

84. A method of reducing triglyceride levels in a subject in need thereof, the method comprising orally administering to the subject a therapeutically effective dose of the oral composition of claim 43.

85.-89. (canceled)

90. A method of providing cognitive support in a subject in need thereof, the method comprising orally administering to the subject a therapeutically effective dose of the oral composition of claim 43.

91. A method of providing mood support in a subject in need thereof, the method comprising orally administering to the subject a therapeutically effective dose of the oral composition of claim 43.

92. A method of improving heart health in a subject in need thereof, the method comprising orally administering to the subject a therapeutically effective dose of the oral composition of claim 43.

93. A method of improving joint health in a subject in need thereof, the method comprising orally administering to the subject a therapeutically effective dose of the oral composition of claim 43.

94. A method of improving eye health in a subject in need thereof, the method comprising orally administering to the subject a therapeutically effective dose of the oral composition of claim 43.

95. A method of providing thermoregulation support in a subject in need thereof, the method comprising orally administering to the subject a therapeutically effective dose of the oral composition of claim 43.

96. A method of providing hormonal balance support in a subject in need thereof, the method comprising orally administering to the subject a therapeutically effective dose of the oral composition of claim 43.

97.-102. (canceled)
